(19) United States
(12) Patent Application Publication
(10) Pub. No.: US 2024/0266003 A1
(43) Pub. Date: Aug. 8, 2024

(54) DETERMINING AND REMOVING INTER-CLUSTER LIGHT INTERFERENCE

(71) Applicant: Illumina, Inc., San Diego, CA (US)

(72) Inventors: Gavin Derek Parnaby, Laguna Niguel, CA (US); Eric Jon Ojard, San Francisco, CA (US)

(21) Appl. No.: 18/434,416

(22) Filed: Feb. 6, 2024

Related U.S. Application Data
(60) Provisional application No. 63/483,428, filed on Feb. 6, 2023.

(51) Int. Cl. G16B 40/20; C12Q 1/6874; G16B 30/10; G16B 30/20

(52) U.S. Cl. CPC G16B 40/20; C12Q 1/6874; G16B 30/10; G16B 30/20

(57) ABSTRACT

This disclosure describes embodiments of methods, systems, and non-transitory computer readable media that accurately estimates the crosstalk from an adjacent cluster of oligonucleotides onto a target cluster of oligonucleotides and removes or reduces the crosstalk emitted by the adjacent cluster of oligonucleotides from the target cluster of oligonucleotides. For instance, the disclosed systems can detect the intensity values for a target cluster and the adjacent cluster. Based on the intensity values of the adjacent cluster, the disclosed systems can determine an inter-cluster-interference metric that estimates the crosstalk emitted from the adjacent cluster. The disclosed systems can remove the inter-cluster-interference metric from the intensity value of the target cluster and generate modified intensity values for the target cluster.

### PRIORITY APPLICATION

The present application claims the benefit of, and priority to, U.S. Provisional Application No. 63/483,428, titled, “DETERMINING AND REMOVING INTER-CLUSTER LIGHT INTERFERENCE,” filed Feb. 6, 2023. The aforementioned application is hereby incorporated by reference in its entirety.

### BACKGROUND

In recent years, biotechnology firms and research institutions have improved hardware and software platforms used for determining a sequence of nucleotide bases (also referred to as “nucleobases”) in a sample. For instance, some existing sequencing machines and sequencing-data-analysis software (together “existing sequencing systems”) determine individual nucleotide bases of nucleic-acid sequences by using conventional Sanger sequencing or by using sequencing-by-synthesis (SBS). When using SBS, existing sequencing systems can monitor thousands, tens of thousands, or more nucleic-acid polymers being synthesized in parallel to detect more accurate nucleotide-base calls. For instance, a camera in SBS platforms can capture images of irradiated fluorescent tags from nucleotide bases incorporated into such synthesized nucleic-acid sequences (often grouped into clusters). After capturing the images, a computing device from the existing systems uses sequencing-data-analysis software to determine nucleotide bases that were detected in a given image based on the light signal captured in the image data. By iteratively incorporating nucleotide bases into the oligonucleotides and capturing images of the emitted light signals in various sequencing cycles, existing sequencing systems can determine the sequence of nucleotide bases present in the samples.

To increase sample throughput and efficiency, existing sequencing systems have grouped clusters of oligonucleotides closer and closer together within wells of flow cells or on other nucleotide-sample slides. As cluster density increases, fluorescence responses (e.g., signals) from one cluster are more likely to interfere with the fluorescence response (or non-response) of neighboring clusters by causing overlapping signals between clusters. Such overlapping signals and light interference are often called spatial crosstalk. Existing sequencing systems attempt to reduce interfering signals by reducing light interference to various components and implementing computation models that estimate and disaggregate interfering responses (e.g., DC offset, noise level, and/or point spread function) from a cluster signal. Unfortunately, the increased density and increased light interference between clusters makes it more difficult to estimate a point spread function (PSF) for a given cluster or section of a nucleotide-sample slide.

As a consequence of nucleotide-sample slides carrying more densely packed clusters, a sequencing device, along with other intensity detecting systems, is more likely to incorrectly determine a cluster is illuminated (instead of not illuminated) because of the spatial crosstalk from a neighboring cluster. The increased crosstalk-along with variations in amplitude and background noise-reduces the accuracy of nucleobase calling based on a signal from a particular cluster. For instance, the increased crosstalk from multiple neighboring clusters can illuminate a given cluster within an image for a given channel. Such indirect illumination within an existing sequencing system can cause the base-calling algorithm to determine an incorrect nucleobase call (e.g., adenine) instead of a correct nucleobase call (e.g., guanine) for the nucleobase incorporated by oligonucleotides of a cluster during a given cycle.

Because crosstalk has placed limits on the accuracy of nucleobase calls by existing sequencing systems, some existing systems have maintained distances among clusters and, consequently, limited the sample and cluster throughput of a sequencing device. As indicated above, when existing sequencing systems increase cluster density on flow cells or other nucleotide-sample slides, quality of the imaging and accuracy of nucleobase calling decreases, resulting in lower data output. To maintain quality imaging and relatively accurate nucleobase calling, some existing sequencing systems limit the number and density of clusters on a nucleotide-sample slide. By avoiding over clustering (e.g., placing too many clusters on a flow cell) and/or under clustering (e.g., placing fewer clusters on a flow cell), existing systems limit nucleotide sequencing to a narrow range of cluster densities on flow cells and reduce data yield.

These, along with additional problems and issues exist in current sequencing systems.

### SUMMARY

This disclosure describes embodiments of methods, non-transitory computer readable media, and systems that can estimate crosstalk of neighboring clusters of oligonucleotides on a target cluster of oligonucleotides (“target cluster”) and remove or reduce the crosstalk from a signal emitted by the target cluster when determining a modified signal for the target cluster. For example, the disclosed systems can detect intensity values for various clusters of oligonucleotides to which labeled nucleotide bases are added. Based on the intensity values for different sets of clusters, the disclosed systems can determine illumination indicators for one or more clusters adjacent to a target cluster. From the illumination indicators and/or other data concerning the adjacent clusters of oligonucleotides (“adjacent clusters”), the disclosed systems determine an inter-cluster-interference metric that estimates light interference of an adjacent cluster on the target cluster. The disclosed systems can further remove the inter-cluster-interference metric from intensity values of the target cluster.

The disclosed systems can utilize such inter-cluster-interference metrics associated with the clusters for a variety of base-calling applications described further below. For example, the disclosed systems can more accurately determine cluster signals and their corresponding nucleobase calls for a given sequencing cycle by (i) removing the crosstalk of neighboring clusters from intensity values of the target cluster when determining the intensity value of the target cluster's signal and (ii) determining a nucleobase call for the target cluster. To increase accuracy and efficiency of nucleobase calling, in some cases, the disclosed systems iteratively determine and remove or reduce crosstalk of adjacent subsets of clusters from target subsets of clusters based on intensity-values ranges for respective clusters.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description will describe various embodiments with additional specificity and detail through the use of the accompanying drawings, which are summarized below.

FIG. 1 illustrates an environment in which a crosstalk-aware-base-calling system can operate in accordance with one or more embodiments of the present disclosure.

FIG. 2 illustrates an overview diagram of the crosstalk-aware-base-calling system generating a modified intensity value for a target cluster by determining and removing an inter-cluster-interference-metric from intensity values of the target cluster in accordance with one or more embodiments of the present disclosure.

FIG. 3 illustrates a diagram demonstrating light interference increases between clusters of oligonucleotides as the distance between clusters of oligonucleotides decreases in accordance with one or more embodiments of the present disclosure.

FIG. 4 illustrates the crosstalk-aware-base-calling system determining illumination indicators based on fluorescent responses in different channels in accordance with one or more embodiments of the present disclosure.

FIGS. 5A-5B illustrate a crosstalk-aware-base-calling system utilizing a linear equalizer system and generating a modified intensity value for a target cluster by determining a nucleobase call and illumination indicators from an adjacent cluster, a signal model for a target cluster, and an inter-cluster-interference metric in accordance with one or more embodiments.

FIG. 6 illustrates an estimated point spread function for intensity values of a signal from a cluster of oligonucleotides in accordance with one or more embodiments of the present disclosure.

FIGS. 7A-7C illustrate effects of light interference between clusters of oligonucleotides and removal of light interference for certain clusters of oligonucleotides in accordance with one or more embodiments of the present disclosure.

FIG. 8A-8B illustrate histograms of intensity values for clusters of oligonucleotides both with light interference from adjacent clusters of oligonucleotides and without light interference from adjacent clusters of oligonucleotides in accordance with one or more embodiments of the present disclosure.

FIG. 9 illustrates a series of acts for generating a modified set of intensity values for a cluster of oligonucleotides using an inter-cluster-interference metric in accordance with one or more embodiments of the present disclosure.

FIG. 10 illustrates a block diagram of an example computing device in accordance with one or more embodiments of the present disclosure.

### DETAILED DESCRIPTION

The disclosure describes one or more embodiments of a crosstalk-aware-base-calling system that determines an inter-cluster-interference metric representing light interference of one cluster of oligonucleotides on a target cluster of oligonucleotides and generating a modified intensity values for the target cluster based on the inter-cluster-interference metric. By determining and removing an inter-cluster-interference metric, the crosstalk-aware-base-calling system disaggregates light interference between clusters. To detect and disaggregate light based on such an inter-cluster-interference metric, in some implementations, the crosstalk-aware-base-calling system determines intensity values for signals from a target cluster and an adjacent cluster of oligonucleotides for a given sequencing cycle. Based on the intensity values of the adjacent cluster, the crosstalk-aware-basing-calling system determines illumination indicators representing whether the adjacent cluster is illuminated during the given sequencing cycle. Based on the illumination indicators, the crosstalk-aware-base-calling system determines an inter-cluster-interference metric estimating light interference from the adjacent cluster on the target cluster. The crosstalk-aware-base-calling system can further subtract (or otherwise remove) the inter-cluster-interference metric from the intensity values of the target cluster's signal to create modified intensity values for the target cluster.

As suggested above, in one or more embodiments, the crosstalk-aware-base-calling system detects intensity values (e.g., wavelength and/or brightness values) for signals emitted by a target cluster and adjacent clusters at a given sequencing cycle. For example, in some cases, the crosstalk-aware-base-calling system detects intensity values from signals emitted by each cluster on a sample-nucleotide slide at a given sequencing cycle-including the clusters that become target and adjacent clusters. In certain embodiments, clusters with higher intensity values are relatively brighter, whereas clusters with lower intensity values are relatively darker. In some cases, the crosstalk-aware-base-calling system leverages the data for the brighter clusters to determine the crosstalk of the brighter clusters on the darker clusters.

Based on detected intensity values, for instance, the crosstalk-aware-base-calling system can determine a subset of illumination indicators for a subset of clusters-including adjacent clusters to a target cluster. In particular, the crosstalk-aware-base-calling system determines nucleobase calls for a subset of clusters (e.g., subset of brighter clusters incorporating adenine) and determines illumination indicators for the subset of clusters from the nucleobase calls. Such illumination indicators identify whether a given cluster is illuminated or emits a fluorescent response in a given channel (e.g., of two or four channels) during a sequencing cycle. and, together with the florescent response in other given channel(s) form data for determining a nucleobase call. In some cases, the illumination indicators together represent illumination of a cluster in multiple channels, such as a first illumination indicator indicating whether a given cluster is illuminated in a first channel during a given sequencing cycle and a second illumination indicator indicating whether the given cluster is illuminated in a second channel during the given sequencing cycle. By contrast, in some cases, illumination indicators can be continuous illumination indicators and indicate a degree to which a given cluster is illuminated in a given channel.

Based on such illumination indicators, as previously suggested, the crosstalk-aware-base-calling system determines an inter-cluster-interference metric (e.g., crosstalk metric). As mentioned above, in some instances, crosstalk indicates how the signal (e.g., brightness) of an adjacent cluster interferes, manipulates, and/or alters the signal of a target cluster. In particular, in one or more embodiments, an inter-cluster-interference metric estimates a degree or extent to which light from an adjacent cluster interferes or modifies light from a target cluster. In some cases, the crosstalk-aware-base-calling system can determine multiple inter-cluster-interference metrics that each estimate light interference from a given adjacent cluster on the target cluster.

Having determined an inter-cluster-interference metric, the crosstalk-aware-base-calling system can utilize the inter-cluster-interference metric to generate modified intensity values for signals emitted by clusters during a sequencing cycle. By leveraging such a metric, the crosstalk-aware-base-calling system can determine the amount of crosstalk between clusters and remove or reduce the crosstalk from a target cluster. To illustrate, in one or more embodiments, during a sequencing cycle, a target cluster can have a relatively dimmer (e.g., lower intensity) signal and a neighboring cluster can have a relatively brighter (e.g., higher intensity) signal. But the brightness of the neighboring cluster's signal can increase the brightness (e.g., intensity) of the target cluster's signal, making it difficult to determine whether the cluster emits light intensity in a particular frequency (e.g., frequency band or spectral band) in a given channel during the sequencing cycle. In some embodiments, the crosstalk-aware-base-calling system can determine an inter-cluster-interference metric based on the illumination indicators and other data concerning an adjacent cluster. Based on the inter-cluster-interference metric, the crosstalk-aware-base-calling system can cancel out (or reduce the effect of) the light emitting from the brighter, adjacent cluster's signal from the target cluster's signal. Thus, in some embodiments, the crosstalk-aware-base-calling system can more accurately determine a target cluster's intensity value in both channels or in each relevant channel during a sequencing cycle based on the inter-cluster-interference metric, which leads to a more accurate nucleobase call of the target cluster.

To increase efficiency and accuracy, in some cases, the crosstalk-aware-base-calling system follows a particular order to determine nucleobase calls and remove crosstalk for clusters. For instance, the crosstalk-aware-base-calling system can (i) identify and determine nucleobase calls for a brightest subset of oligonucleotide clusters emitting signals within a top intensity-value range (e.g., top 10% brightest) and (ii) further determine inter-cluster-interference metrics estimating light interference of the brightest subset of oligonucleotide clusters on a next brightest subset of oligonucleotide clusters emitting signals within a second intensity-value range (e.g., top 20-30% brightest). As explained further below, the crosstalk-aware-base-calling system can likewise perform further iterations of determining crosstalk based on additional intensity-value ranges. In the alternative to ordering nucleobase calling and crosstalk removal according to intensity-value ranges, in some embodiments, the crosstalk-aware-base-calling system can use signal-to-noise ratio (SNR) metrics to order nucleobase calling and crosstalk removal for clusters.

The crosstalk-aware-base-calling system provides several advantages over conventional sequencing platforms. In particular, the crosstalk-aware-base-calling system can disaggregate the light intensity comprising a cluster signal's intensity and noise from other sources, improve the accuracy of nucleobase calling, and increase the efficiency of flow cells or nucleotide-sample slides during sequencing cycles. As mentioned, the crosstalk-aware-base-calling system can receive a signal with an unmodified intensity value from the target cluster, where the unmodified intensity value of the signal from the target cluster comprises the signal from the target cluster, crosstalk (e.g., noise) from adjacent clusters, and other sources of noise (e.g., background noise or intensity fluctuations). The crosstalk-aware-base-calling system can disaggregate the light intensity comprised of the target signal and noise. In particular, the crosstalk-aware-base-calling system can determine an inter-cluster-interference metric that estimates the light interference from an adjacent cluster on a target cluster. The inter-cluster-interference metric estimates the crosstalk (e.g., interfering light) of the adjacent cluster from the composite parts (e.g., background noise, intensity value of the target cluster's signal, crosstalk). Once estimated, the crosstalk-aware-base-calling system can remove or reduce the crosstalk by removing the inter-cluster-interference metric from the target cluster's signal. While existing sequencing systems often fail to determine whether a target cluster is emitting a signal based on variations in background noise, crosstalk, and/or amplitude, the inter-cluster-interference metric allows the crosstalk-aware-base-calling system to accurately disaggregate crosstalk from background noise and/or amplitude. Unlike existing sequencing systems, upon determining the degree and source of crosstalk, in one or more embodiments, the crosstalk-aware-base-calling system can remove the crosstalk from the signal of the affected cluster. By removing the inter-cluster-interference metric from the target cluster's signal, the crosstalk-aware-base-calling system can generate a modified signal for the target cluster. Thus, by removing the crosstalk from the target cluster's signal and generating a more accurate signal for the target cluster, the crosstalk-aware-base-calling system can more accurately and confidently determine a nucleobase call for a target cluster.

In addition to detecting and disaggregating part of a signal composed of noise and light, the crosstalk-aware-base-calling system improves nucleobase calling accuracy. In particular, the crosstalk-aware-base-calling system can determine an inter-cluster-interference metric estimating light interference from an adjacent cluster on a target cluster and remove the inter-cluster-interference metric from intensity values of the target cluster's signal. The resulting modified intensity values represent a more accurate and/or purer signal for the target cluster. Based on the more accurate or purer cluster signal, the crosstalk-aware-base-calling system can likewise determine more accurate or confident nucleobase calls for the target cluster-without or with minimal crosstalk interfering with the signal determining the nucleobase calls. For example, the crosstalk-aware-base-calling system can determine that modified intensity values for a target cluster fall within the intensity-value boundaries of one nucleobase instead of another nucleobase or improve the confidence score (e.g., QUAL score) of a nucleobase call with a low-quality score.

In addition to improved nucleotide-base calls and disaggregating crosstalk from cluster signals, the crosstalk-aware-base-calling system improves the efficiency with which a sequencing system performs nucleotide sequencing. By determining and removing inter-cluster-interference metrics and improving the signal of target clusters, the crosstalk-aware-base-calling system facilitates more densely grouped clusters on a nucleotide-sample slide. Unlike the more limited and less densely grouped clusters of existing sequencing systems, the crosstalk-aware-base-calling system introduces a model that removes or reduces crosstalk and facilitates more densely grouped clusters and higher throughput on a sequencing device. By determining and removing inter-cluster-interference metrics, therefore, the crosstalk-aware-base-calling system can sequence the nucleotide sequence of more genomic samples with improved accuracy over existing sequencing systems that cannot effectively adjust for the crosstalk of densely grouped clusters.

As illustrated by the foregoing discussion, the present disclosure utilizes a variety of terms to describe features and advantages of the crosstalk-aware-base-calling system. Additional detail is now provided regarding the meaning of such terms. For example, as used herein, the term “nucleotide-sample slide” refers to a plate or slide comprising oligonucleotides for sequencing nucleotide segments for samples. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to adaptor sequences.

Relatedly, as used herein, the term “section of a nucleotide-sample slide” (or “nucleotide-sample slide section”) refers to an area that is part of a nucleotide-sample slide. In particular, a section of a nucleotide-sample slide can refer to a discrete portion of a nucleotide-sample slide that differs from other portions of the nucleotide-sample slide. For instance, a section of a nucleotide-sample slide can include a well (e.g., a nano-well) of a patterned flow cell or a discrete subsection of a non-pattered flow cell (e.g., a subsection corresponding to a cluster). In some cases, a section of a nucleotide-sample slide includes a tile or a sub-tile having clusters of the same or similar oligonucleotide growing in parallel.

Additionally, as used herein, the term “labeled nucleotide base” refers to a nucleotide base having a fluorescent or light-based indicator or fluorescent dye indicator of the classification of the nucleotide base. In particular, a labeled nucleotide base can refer to a nucleotide base that incorporates a fluorescent or light-based indicator or fluorescent dye indicator to identify the type of base (e.g., adenine, cytosine, thymine, or guanine). For example, in one or more embodiments, a labeled nucleotide base includes a nucleotide base having a fluorescent tag that emits a signal that either by itself or together with another fluorescent tag identifies the base type. Accordingly, a nucleotide base may be identified by a mixture of dyes (or a mixture of fluorescent tags) that together indicate the nucleobase type (e.g., “ON”/“ON” illumination indicators). Based on intensity values for a signal emitted by labeled nucleotide bases in a cluster of oligonucleotides, such as signals in 16 quadrature amplitude modulation (QAM) or pulse amplitude modulation (PAM) 4 format, the type of base (e.g., adenine, cytosine, thymine, or guanine) can be determined in certain embodiments of the crosstalk-aware-base-calling system.

Moreover, as used herein, the term “cluster of oligonucleotides” refers to a grouping containing several identical deoxyribonucleic acid (DNA) fragments bound to the surface of a flow cell. For instance, in some embodiments, a cluster of oligonucleotides can be made up of a template DNA strand that has been clonally amplified through bridge amplification.

Further, as used herein the term “signal” refers to a signal emitted, reflected, or otherwise communicated from a labeled nucleotide base or a group of labeled nucleotide bases (e.g., labeled nucleotide bases added to a cluster of oligonucleotides). In particular, a signal can refer to a signal indicating the type of base. For example, a signal can include a light signal emitted or reflected from a fluorescent tag of a nucleotide base or fluorescent tags of multiple nucleotide bases incorporated into oligonucleotides. As indicated above, a nucleobase incorporated into a cluster may (in response to a laser) likewise emit a signal that can be identified as a mixture of dyes (or a mixture of fluorescent tags) that together indicate the nucleobase type (e.g., a cluster with “ON”/“ON” illumination indicators). In some implementations, the crosstalk-aware-base-calling system triggers the signal through an external stimulus, such as a laser or other light source. In some cases, the crosstalk-aware-base-calling system triggers the signal through some internal stimuli. Further, in some embodiments, the crosstalk-aware-base-calling system observes the signal using a filter applied when capturing an image of the nucleotide-sample slide (e.g., section of the nucleotide-sample slide). As suggested above, in certain instances, a signal includes an aggregate of the signals provided by each labeled nucleotide base added to individual oligonucleotides in a cluster of oligonucleotides.

As used herein, the term “intensity value” refers to a value indicating a characteristic or attribute of a signal emitted, reflected, or otherwise communicated from a labeled nucleotide base or a group of labeled nucleotide bases from a cluster of oligonucleotides. In particular, an intensity value can refer to a value associated with a color intensity (e.g., wavelength) or a light intensity (e.g., brightness). In some cases, the crosstalk-aware-base-calling system captures several images of a cluster of oligonucleotides with labeled nucleotide bases using different filters (or intensity channels). Thus, an intensity value of a signal can correspond to the intensity of the signal as observed through a particular filter.

As used herein, the term “illumination indicator” refers to an indicator of whether a cluster of oligonucleotides is illuminated by or emits an intensity of light in a particular frequency band during a sequencing cycle. In particular, an illumination indicator represents whether (or a degree to which) a cluster of oligonucleotides (i) comprises labeled nucleotides emitting a particular intensity of light in a particular frequency (e.g., frequency band) to become illuminated (e.g., on or active) or (ii) does not comprise labeled nucleotide bases such that it is not illuminated (e.g., off or inactive) by a particular intensity of light in a particular frequency (e.g., frequency band) in an intensity channel during sequencing. In some cases, an illumination indicator can take a couplet format. For example, if a cluster of oligonucleotides incorporates nucleobases with fluorescent tags or other labels that (in response to a light or laser) illuminate or emit a light intensity in a particular frequency (e.g., frequency band) of light in a channel during a sequencing cycle, the “on” or “illuminated” status for an illumination indicator can be represented by a one. Conversely, if a cluster of oligonucleotides does not incorporate (or incorporates too few) nucleobases with fluorescent tags or other labels that (in response to a light or laser) illuminate or emit light intensity in a particular frequency in a particular frequency (e.g., frequency band) in a channel during a sequencing cycle, the “off” or “unilluminated” status for an illumination indicator can be represented by a zero. To illustrate, [1,1] can indicate that an illumination indicator for a cluster of oligonucleotides is illuminated in two different channels. While the description and figures depict illumination indicators in different channels (e.g., two channels or four channels), the crosstalk-aware-base-calling system can detect signals from clusters concurrently in such different channels.

By contrast, if a polyclonal cluster of oligonucleotides incorporates nucleobases with different fluorescent tags or other labels that (in response to a light or laser) illuminate or emit light within different spectral bands in a given channel during a sequencing cycle, the status for an illumination indicator would not be entirely “on” or “off” (or not be entirely “illuminated” or “unilluminated”). In some cases, such a mixed signal from a polyclonal cluster of oligonucleotides is filtered out and discarded based on intensity-value boundaries for different types of nucleobases.

While this disclosure frequently uses illumination indicators in the form of “on” or “off” (or a corresponding “1” or “0”), an illumination indicator can be particular to a channel and is not designed to indicate a presence or absence of background noise or other light. Accordingly, an “off” or “0” indicator does not indicate an absence of light, but rather an estimate that a particular cluster did not incorporate (or incorporates too few) nucleobases with fluorescent tags or another label that (in response to a light or laser) illuminate or emit light intensity in a particular frequency (e.g., frequency band) in a particular channel during a sequencing cycle. Accordingly, an illumination indicator can take other formats. In the alternative to a couplet format, in some embodiments, an illumination indicator may be continuous and represent a degree to which a given cluster is illuminated during a sequencing cycle. Such a continuous illumination indicator, for example, can take the form of a metric or score (e.g., between 0 and 1) indicating a degree to which a cluster is illuminated by light emitted from a particular type of nucleotide incorporated into the cluster during a sequencing cycle.

Additionally, as used herein, the term “inter-cluster-interference metric” refers to a measure or quantification of light from one cluster of oligonucleotides interfering or modifying light from another cluster of oligonucleotides. In particular, an inter-cluster-interference metric can refer to the degree, amount, and/or extent of interference of a light signal from one cluster of oligonucleotides on another cluster of oligonucleotides.

As used herein, the term “nucleotide-base call” refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate a determination or prediction of the type of nucleobase that has been incorporated within an oligonucleotide on a nucleotide-sample slide (e.g., read-based nucleobase calls). In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). As suggested above, a single nucleobase call can be an adenine (A) call, a cytosine (C) call, a guanine (G) call, a thymine (T) call, or an uracil (U) call.

Additionally, as used herein, the term “sequencing cycle” (or “cycle”) refers to an iteration of adding or incorporating a nucleotide base to an oligonucleotide or an iteration of adding or incorporating nucleotide bases to oligonucleotides in parallel. In particular, a cycle can include an iteration of taking an analyzing one or more images with data indicating individual nucleotide bases added or incorporated into an oligonucleotide or to oligonucleotides in parallel. Accordingly, cycles can be repeated as part of sequencing a nucleic-acid polymer. For example, in one or more embodiments, each sequencing cycle involves either single reads in which DNA or RNA strands are read in only a single direction or paired-end reads in which DNA or RNA strands are read from both ends. Further, in certain cases, each sequencing cycle involves a camera taking an image of the nucleotide-sample slide or multiple sections of the nucleotide-sample slide to generate image data for determining a particular nucleotide base added or incorporated into particular oligonucleotides. Following the image capture stage, a sequencing system can remove certain fluorescent labels from incorporated nucleotide bases and perform another sequencing cycle until the nucleic-acid polymer has been completely sequenced. In one or more embodiments, a sequencing cycle includes a cycle within a Sequencing By Synthesis (SBS) run.

Additionally, as used herein, the term “nucleotide-base-call data” refers to a digital file, image data, or other digital information indicating individual nucleotide bases or the sequence of nucleotide bases for a nucleic-acid polymer. In particular, nucleotide-base-call data can include intensity values (e.g., color or light intensity values for individual clusters) from images taken by a camera of a nucleotide-sample slide or other data that indicate individual nucleotide bases or the sequence of nucleotide bases for a nucleic-acid polymer. In addition, or in the alternative to intensity values, the nucleotide-base-call data may include chromatogram peaks or electrical current changes indicating individual nucleobases in a sequence. Additionally, in some embodiments, nucleotide-base-call data includes individual nucleotide-base calls identifying the individual nucleotide bases (e.g., A, T, C, or G). For example, nucleotide-base-call data can comprise data for nucleotide-base calls in a sequence for a nucleic-acid polymer, the number of nucleotide-base calls corresponding to a particular base (e.g., adenine, cytosine, thymine, or guanine), as organized in a digital file, such as a Binary Base Call (BCL) file. Further, nucleotide-base call data can include error/accuracy information, such as a quality metric associated with each nucleotide-base call. In some embodiments, nucleotide-base-call data comprises information from a sequencing device that utilizes sequencing by synthesis (SBS).

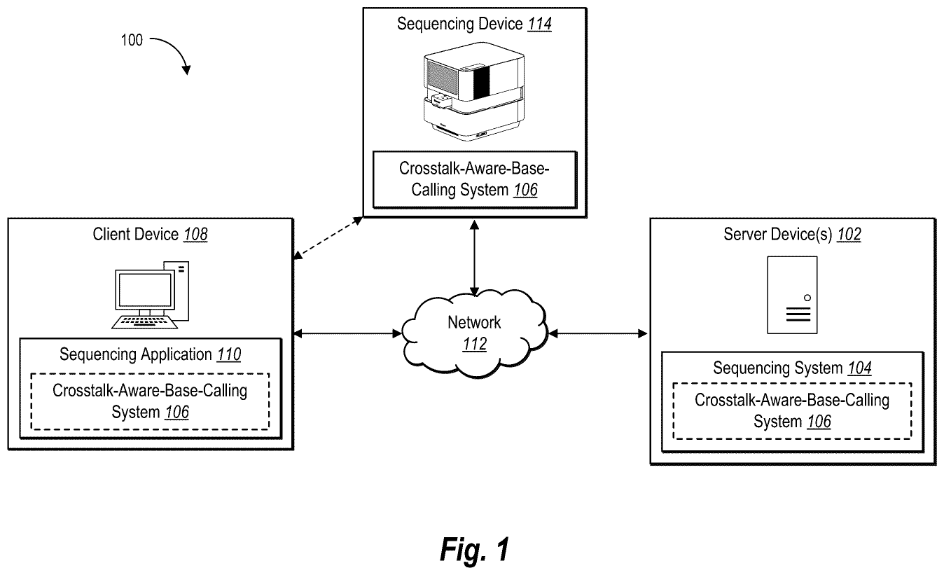

Additional detail will now be provided regarding the crosstalk-aware-base-calling system in relation to illustrative figures portraying example embodiments and implementations of the crosstalk-aware-base-calling system. For example, FIG. 1 illustrates a schematic diagram of a system environment (or “environment”) 100 in which the crosstalk-aware-base-calling system 106 operates in accordance with one or more embodiments. As illustrated, the environment 100 includes one or more server device(s) 102 connected to a user client device 108 and a sequencing device 114 via a network 112. While FIG. 1 shows an embodiment of the crosstalk-aware-base-calling system 106, alternative embodiments and configurations are possible.

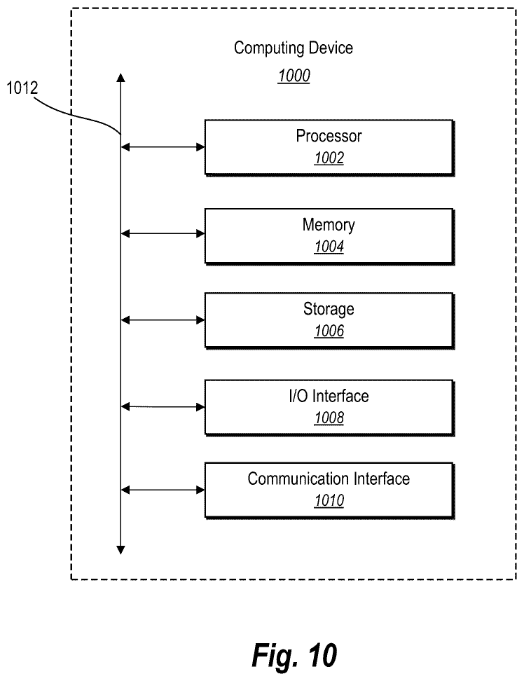

As further shown in FIG. 1, the server device(s) 102, the user client device 108, and the sequencing device 114 are connected via the network 112. Each of the components of the environment 100 can communicate via the network 112. The network 112 comprises any suitable network over which computing devices can communicate. Example networks are discussed in additional detail below in relation to FIG. 10.

As shown in FIG. 1, the environment 100 includes the sequencing device 114. The sequencing device 114 comprises a device for sequencing a whole genome or other nucleic-acid polymer. In some embodiments, the sequencing device 114 analyzes samples to generate data utilizing computer implemented methods and systems described herein either directly or indirectly on the sequencing device 114. In one or more embodiments, the sequencing device 114 utilizes Sequencing By Synthesis (SBS) to sequence whole genomes or other nucleic-acid polymers. As shown, in some embodiments, the sequencing device 114 bypasses the network 112 and communicates directly with the user client device 108.

As further depicted by FIG. 1, the environment 100 includes the server device(s) 102. The server device(s) 102 may generate, receive, analyze, store, receive, and transmit electronic data, such as data for sequencing nucleic-acid polymers. The server device(s) 102 may receive data from the sequencing device 114. For example, the server device(s) 102 may gather and/or receive sequencing data including nucleotide-base call data, quality data, and other data relevant to sequencing nucleic-acid polymers. The server device(s) 102 may also communicate with the user client device 108. In particular, the server device(s) 102 can send read data, nucleic-acid polymer sequences, error data, and other information to the user client device 108. In some embodiments, the server device(s) 102 comprise distributed servers, where the server device(s) 102 include a number of server devices distributed across the network 112 and located in different physical locations. The server device(s) 102 can comprise a content server, an application server, a communication server, a web-hosting server, or another type of server.

As further shown in FIG. 1, the server device(s) 102 can include the sequencing system 104. Generally, the sequencing system 104 analyzes sequencing data received from the sequencing device 114 to determine nucleotide sequences for whole genomic samples or other nucleic-acid polymers. For example, the sequencing system 104 can receive raw data (e.g., base-call data for nucleotide reads) from the sequencing device 114 and determine a nucleic acid sequence for a genomic sample. To illustrate, the sequencing system 104 can receive data for nucleotide reads from the sequencing device 114, and the sequencing system 104 generates variant calls (or other nucleobase calls) for a genomic sample from the nucleotide reads. In some embodiments, the sequencing system 104 determines the sequences of nucleotide bases in DNA and/or RNA.

As further illustrated in FIG. 1, the sequencing device 114 includes the crosstalk-aware-base-calling system 106. Generally, the crosstalk-aware-base-calling system 106 determines an inter-cluster-interference metric to modify or correct a signal for estimated light interference from adjacent clusters on a target cluster. More specifically, in some embodiments, the crosstalk-aware-base-calling system 106 detects intensity values for a target cluster and an adjacent cluster in a given sequencing cycle. The crosstalk-aware-base-calling system 106 determines a nucleobase call and illumination indicators for the adjacent cluster. The crosstalk-aware-base-calling system 106 further determines an inter-cluster-interface metric for crosstalk of the adjacent cluster on the target cluster. The crosstalk-aware-base-calling system 106 further generates a modified intensity value for the target cluster by removing the inter-cluster-interference metric from intensity values for the target cluster.

The environment 100 illustrated in FIG. 1 further includes the user client device 108. The user client device 108 can generate, store, receive, and send digital data. In particular, the user client device 108 can receive sequencing data from the sequencing device 114. Furthermore, the user client device 108 may communicate with the server device(s) 102 to receive nucleotide-base calls, nucleotide sequences, and variant call files. The user client device 108 can present sequencing data to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, smartphones, etc. Additional details with regard to the user client device 108 are discussed below with respect to FIG. 10.

As further illustrated in FIG. 1, the user client device 108 includes a sequencing application 110. The sequencing application 110 may be a web application or a native application on the user client device 108 (e.g., a mobile application, desktop application, etc.). The sequencing application 110 can comprise instructions that (when executed) cause the user client device 108 to receive or request data from the crosstalk-aware-base-calling system 106 and present sequencing data. Furthermore, the sequencing application 110 can comprise instructions that (when executed) cause the user client device 108 to provide a graphical visualization of a read pileup or read alignment for nucleotide reads for a genomic sample.

As further illustrated in FIG. 1, the crosstalk-aware-base-calling system 106 may be located on the user client device 108 as part of the sequencing application 110. As illustrated, in some embodiments, the crosstalk-aware-base-calling system 106 is implemented by (e.g., located entirely or in part on) the user client device 108. In yet other embodiments, the crosstalk-aware-base-calling system 106 is implemented by one or more other components of the environment 100. In particular, the crosstalk-aware-base-calling system 106 can be implemented in a variety of different ways across the server device(s) 102, the user client device 108, and the sequencing device 114. In one example, the crosstalk-aware-base-calling system 106 is located in part on the sequencing device 114 and also the server device(s) 102. In particular, the crosstalk-aware-base-calling system 106 can determine an inter-cluster-interference metric for crosstalk of the adjacent cluster on the target cluster on the sequencing device 114 and modify the intensity values of the target cluster by removing the inter-cluster-interference metric as part of the server device(s) 102.

Though FIG. 1 illustrates the components of environment 100 communicating via the network 112, in some embodiments, the components of environment 100 communicate directly with each other, bypassing the network. For instance, and as previously mentioned, the user client device 108 can communicate directly with the sequencing device 114. Additionally, the user client device 108 can communicate directly with the crosstalk-aware-base-calling system 106, bypassing the network 112. Moreover, the crosstalk-aware-base-calling system 106 can access one or more databases housed on the server device(s) 102 or elsewhere in the environment 100.

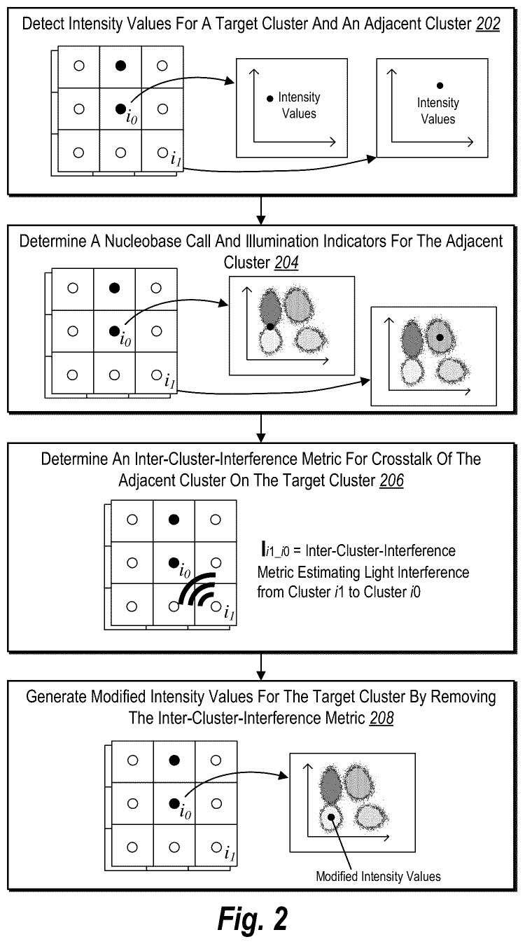

The following paragraphs provide further details concerning the crosstalk-aware-base-calling system 106. In accordance with one or more embodiments, FIG. 2 depicts an overview of the crosstalk-aware-base-calling system 106 generating an inter-cluster-interface metric and modifying an intensity value for a target cluster. As an overview of FIG. 2, the crosstalk-aware-base-calling system 106 performs a series of acts that includes an act 202 of detecting intensity values for a target cluster and an adjacent cluster, an act 204 of determining a nucleobase call and illumination indicators for the adjacent cluster, an act 206 of determining an inter-cluster-interference metric for crosstalk of the adjacent cluster on the target cluster, and an act 208 of generating a modified intensity value for the target cluster by removing the inter-cluster-interference metric.

As just mentioned, FIG. 2 illustrates the act 202 of detecting intensity values for the target cluster and the adjacent cluster. In some embodiments, the crosstalk-aware-base-calling system 106 may detect a set of intensity values for the target cluster and a set of intensity values for the adjacent cluster through laser (e.g., light) excitation and imaging. During a sequencing cycle, the crosstalk-aware-base-calling system 106 can direct a light source with a specified wavelength at a nucleotide-sample slide (or portion of the nucleotide-sample slide) and capture an image of the clusters within the nucleotide-sample slide emitting a signal. In some embodiments, the crosstalk-aware-base-calling system 106 captures multiple images of clusters emitting signals. For instance, the crosstalk-aware-base-calling system 106 can capture multiple images using various filter or intensity channels. To illustrate, in some embodiments, the crosstalk-aware-base-calling system 106 utilizes a two-channel implementation by capturing two images of a section of the nucleotide-sample slide per sequencing cycle. In particular, the crosstalk-aware-base-calling system 106 captures a first image using a first filter and captures a second image using a second filter. The first and second images can capture the intensity of the emitted signal from the target cluster and the adjacent cluster that corresponds to the filter.

The crosstalk-aware-base-calling system 106 can implement sequencing runs, however, using alternative channel-based approaches. In some implementations, the crosstalk-aware-base-calling system 106 utilizes a four-channel implementation and captures four different images of the section of the flow cell. Similar to the two-channel implementation, the crosstalk-aware-base-calling system 106 can capture each image for the four-channel implementation using a different filter. Each image can capture an intensity of the emitted signal based on the filter used for that image. Thus, in some cases, each of the four images depicts the emitted signal with a different intensity. Additionally, the crosstalk-aware-base-calling system 106 can utilize a single channel implementation and capture one image of the section of the nucleotide-sample slide and using a specific filter capture the intensity of the emitted signal. In other embodiments, the crosstalk-aware-base-calling system 106 can utilize a one-channel implementation and capture one image (or a three-channel implementation and capture three images) of the section of the nucleotide-sample slide and capture the intensity value of the emitted signal by utilizing a particular filter.

Based on the captured images of the intensity (e.g., color intensity and/or light intensity) of signals emitted by the target cluster and the adjacent cluster, the crosstalk-aware-base-calling system 106 can measure the intensity of the signals of the target cluster and the adjacent cluster and provide intensity values (e.g., wavelength and/or brightness) for the signals of the target cluster and the adjacent cluster. For example, while utilizing two intensity channels, the crosstalk-aware-base-calling system 106 can measure the wavelength of the signals emitted by the target cluster and the adjacent cluster in the first channel and the second channel.

As further indicated in FIG. 2, the crosstalk-aware-base-calling system 106 can perform an act 204 of determining a nucleobase call and illumination indicators for the adjacent cluster. As previously mentioned, the emitted signals of the cluster can indicate the type of nucleotide base. For instance, in some embodiments, the crosstalk-aware-base-calling system 106 analyzes the intensity values for signals from the given cluster in both channels or each of multiple channels (e.g., concurrently) to determine the nucleobase call. In some embodiments, based on the intensity values of the signal of the cluster in each channel, the crosstalk-aware-base-calling system 106 can calculate, utilizing an expectation maximization and Gaussian probability distributions, the probability that the signal falls within the intensity-value boundaries of a certain base (A, C, G, or T). The crosstalk-aware-base-calling system 106 can then call the nucleobase incorporated into the cluster by selecting the intensity-value boundaries of the nucleobase with the highest probability. For example, based on the intensity values emitted by the signal of the cluster, the crosstalk-aware-base-calling system 106 can determine that the intensity-values boundaries of the nucleobase with the highest probability for the cluster is adenine (A).

After determining the nucleobase call for the cluster, in some embodiments, the crosstalk-aware-base-calling system 106 determines the illumination indicators for the cluster. Based on the nucleobase call, for instance, the crosstalk-aware-base-calling system 106 can decide whether the cluster was “on” (e.g., illuminated or actively emitting light intensity in a particular frequency) or off (e.g., unilluminated or not emitting light intensity in a particular frequency) in a given intensity channel during a sequencing cycle. For example, if a nucleobase call for the cluster is adenine (A), the crosstalk-aware-base-calling system 106 can determine that the first channel signal and the second channel signal of the cluster was “on” (or that the cluster emitted light in both the first and second channel) during the sequencing cycle.

While the previous embodiment describes the crosstalk-aware-base-calling system 106 determining a nucleobase call before determining illumination indicators, in some embodiments, the crosstalk-aware-base-calling system 106 can perform these acts in reverse order. For example, in some embodiments, the crosstalk-aware-base-calling system 106 can determine whether the illumination indicators were “on” or “off” for a given channel during a sequencing cycle and based on the illumination indicators, determine a nucleobase call for the cluster.

In some cases, the crosstalk-aware-base-calling system 106 can represent the status of the illumination indicator within the intensity channel for the adjacent cluster as a set of illumination indicators as a couplet. For instance, in some embodiments, the crosstalk-aware-base-calling system 106 determines an adenine (A) nucleobase call for an adjacent cluster and, consequently, determines the corresponding illumination indicators for the adjacent cluster in two different channels as On/On or [1, 1]. As indicated by FIG. 2, in some embodiments, illumination indicators for nucleobase calls of cytosine (C), thymine (T), and guanine (G) can be represented as On/Off or [1, 0], Off/On or [0, 1], and Off/Off or [0, 0], respectively.

As further shown in FIG. 2, after determining a nucleobase call and set of illumination indicators for the adjacent cluster, the crosstalk-aware-base-calling system 106 performs the act 206 of determining an inter-cluster-interface metric for crosstalk of the adjacent cluster on the target cluster. For example, the crosstalk-aware-base-calling system 106 determines the inter-cluster-interference metric based on a set of illumination indicators for the adjacent cluster. As described further below, in some embodiments, the crosstalk-aware-base-calling system 106 determines the inter-cluster-interference metric based on an amplitude of the adjacent cluster, the set of illumination indicators encoded for the adjacent cluster, and the estimated point spread function response from the location adjacent cluster to the location of target cluster. Based on the estimated amplitude, illumination indicators, and point spread function of the adjacent cluster, the crosstalk-aware-base-calling system 106 can measure the amount of crosstalk (e.g., light interference) from the adjacent cluster on the target cluster.

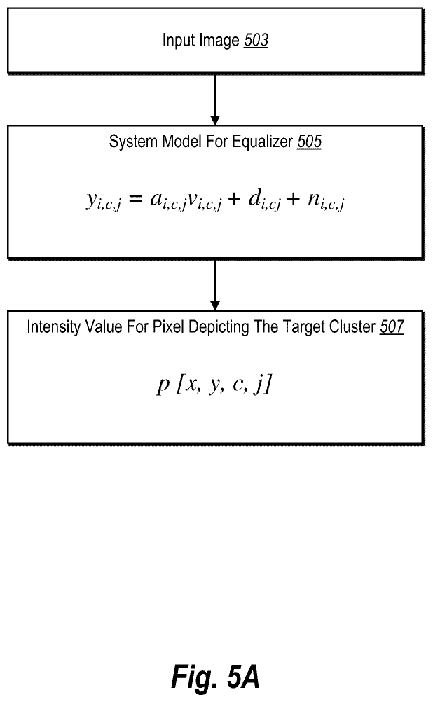

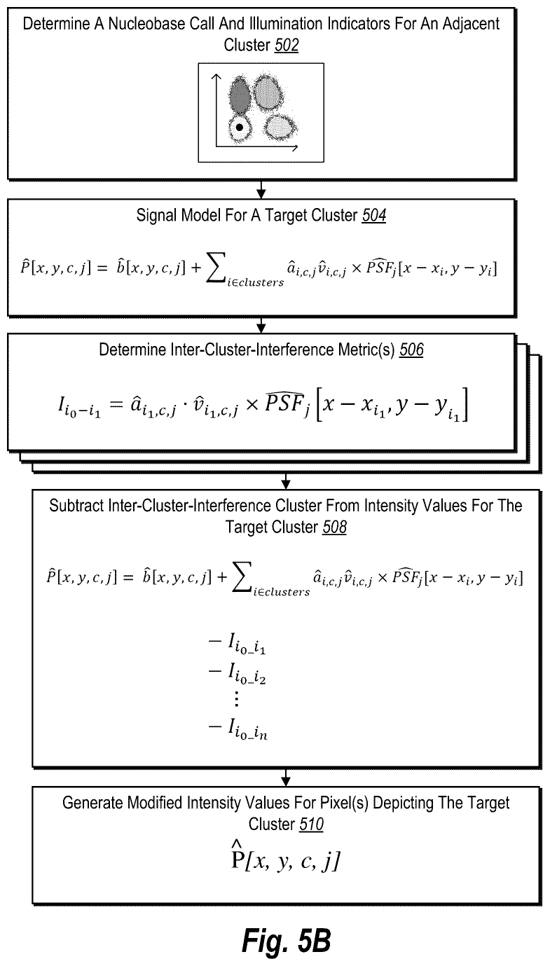

In some cases, the crosstalk-aware-base-calling system 106 utilizes the inter-cluster-interference metric as part of a function to subtract the crosstalk from the target cluster. FIG. 5B and corresponding paragraphs below provide further detail about how the crosstalk-aware-base-calling system 106 estimates and utilizes an amplitude (â_{i1,c,j}) of the adjacent cluster, illumination indicators for the adjacent cluster (and the point spread function response from the adjacent cluster to the target cluster (\hat{v}_{i1,c,j}) in accordance with one or more embodiments to determine an inter-cluster-interference metric (I_{i0_i1}) estimating light interference from the adjacent cluster on the target cluster. In some embodiments, the crosstalk can be modeled as light interference on target clusters. In other embodiments, the crosstalk can be modeled as light interference on pixels associated with a target cluster.

After determining the inter-cluster-interference metric, the crosstalk-aware-base-calling system 106 performs an act 208 of generating modified intensity values for the target cluster by removing the inter-cluster-interference metric. Based on the modified intensity values of the target cluster, the crosstalk-aware-base-calling system 106 can make a more accurate nucleobase call for the target cluster. For example, the crosstalk-aware-base-calling system 106 can determine that the modified intensity values of the target cluster lead to a guanine (G) nucleobase call, whereas the unmodified intensity values for the target cluster initially resulted in a nucleobase call for cytosine (C).

As previously mentioned, the crosstalk-aware-base-calling system 106 can follow a particular cluster order to determine illumination indictors and crosstalk in a given sequencing cycle. For instance, the crosstalk-aware-base-calling system 106 can identify a first subset of oligonucleotide clusters that emit the brightest signals within a top intensity-value range (e.g., top 10% brightest). The crosstalk-aware-base-calling system 106 subsequently determines (i) nucleobase calls for the first subset of oligonucleotide clusters and (ii) inter-cluster-interference metrics estimating interference of clusters from the first subset of oligonucleotide clusters on a second subset of oligonucleotide clusters that emit signals within a second intensity-value range (e.g., top 20-30% brightest). The remaining order of clusters can follow a same or similar pattern based on additional intensity-value ranges. In some cases, for example, the crosstalk-aware-base-calling system 106 determines (i) nucleobase calls for the second subset of oligonucleotide clusters and (ii) inter-cluster-interference metrics estimating interference of clusters from the second subset of oligonucleotide clusters on a third subset of oligonucleotide clusters that emit signals within a third intensity-value range (e.g., top 30-40% brightest).

As just described, the crosstalk-aware-base-calling system 106 can determine nucleobase calls, illumination indictors, and crosstalk in a given sequencing cycle in an order based on intensity-value ranges. As an alternative to using intensity-value ranges, the crosstalk-aware-base-calling system 106 may (i) identify a first subset of oligonucleotide clusters based on DC offset and amplitude for signals emitted by clusters and (ii) inter-cluster-interference metrics estimating interference of clusters from the first subset of oligonucleotide clusters on a second subset of oligonucleotide clusters that emit signals. For instance, the crosstalk-aware-base-calling system 106 can (i) identify a first subset of oligonucleotide clusters that exhibit a combination of DC offset and amplitude within a first threshold difference of the received intensity value for a given cluster and (ii) identify a second subset of oligonucleotide clusters that exhibit a combination of DC offset and amplitude within a second threshold difference of the received intensity value for the given cluster.

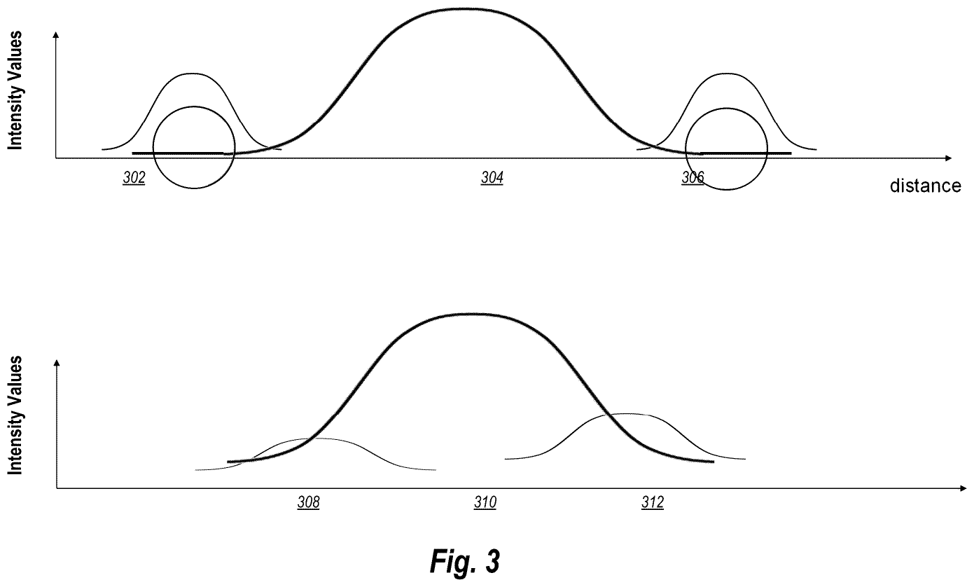

FIG. 2 provides an overview of acts performed by the crosstalk-aware-base-calling system 106 as part of generating modified intensity values for the target cluster by utilizing the inter-cluster interface metric to remove or reduce crosstalk from the adjacent cluster on the target cluster. In accordance with one or more embodiments, FIG. 3 illustrates an example of crosstalk (e.g., light interference) increasing between clusters as the distance between clusters decreases. In particular, FIG. 3 depicts a one-dimensional cross section of a two-dimensional nucleotide-sample slide containing three clusters of oligonucleotides to show how distance between clusters affects crosstalk between clusters.

As previously discussed, some existing sequencing systems limit the number and density of clusters of oligonucleotides on a flow cell to maintain accurate nucleobase calling. As shown in FIG. 3, when sufficient distance exists between an adjacent cluster of oligonucleotides 302, a center cluster of oligonucleotides 304, and an adjacent cluster of oligonucleotides 306, the signal of the center cluster of oligonucleotides 304 with relatively higher intensity values does not overlap (or minimally overlaps) with the signals of the adjacent cluster of oligonucleotides 302 and the adjacent cluster of oligonucleotides 306 with relatively lower intensity values.

Because of the relatively little overlap between signals of the adjacent cluster of oligonucleotides 302, the center cluster of oligonucleotides 304, and the adjacent cluster of oligonucleotides 306, existing sequencing systems can more easily detect the interference from the signal of the center cluster of oligonucleotides 304 on the intensity values of signals emitted by the adjacent cluster of oligonucleotides 302 and the adjacent cluster of oligonucleotides 306. With more accurate or purer intensity values for the adjacent cluster of oligonucleotides 302 and the adjacent cluster of oligonucleotides 306, the crosstalk-aware-base-calling system 106 can more accurately make a nucleobase call and determine whether the adjacent cluster of oligonucleotides 302 and the adjacent cluster of oligonucleotides 306 are “on” (e.g., illuminated or emitting light intensity in a particular frequency) or “off” (e.g., unilluminated or not emitting light intensity in a particular frequency) in a certain intensity channels during a sequencing cycle.

As further depicted in FIG. 3, as density increases between clusters of oligonucleotides, it becomes more difficult to accurately determine the intensity values and nucleobase calls for clusters of oligonucleotides emitting relatively lower (e.g., relatively dimmer) intensity values because the light interference from clusters of oligonucleotides emitting relatively higher (e.g., relatively brighter) intensity values affect the intensity values of the dimmer clusters of oligonucleotides.

As shown in FIG. 3, for example, the decreased distance between an adjacent cluster of oligonucleotides 308, a center cluster of oligonucleotides 310, and an adjacent cluster of oligonucleotides 312 causes increased crosstalk interfering with the relatively lower intensity values of the adjacent cluster of oligonucleotides 308 and the adjacent cluster of oligonucleotides 312. More specifically, the light signal emitted from the center cluster of oligonucleotides 310 interferes with or makes it more difficult to detect intensity values of the adjacent cluster of oligonucleotides 308 and the adjacent cluster of oligonucleotides 312. As a consequence of the light interference and increased likelihood of detecting intensity values of the center cluster of oligonucleotides 310 incorrectly attributed to the adjacent cluster of oligonucleotides 308 and the adjacent cluster of oligonucleotides 312, existing sequencing systems often cannot accurately make a nucleobase call for the adjacent cluster of oligonucleotides 308 and the adjacent cluster of oligonucleotides 312.

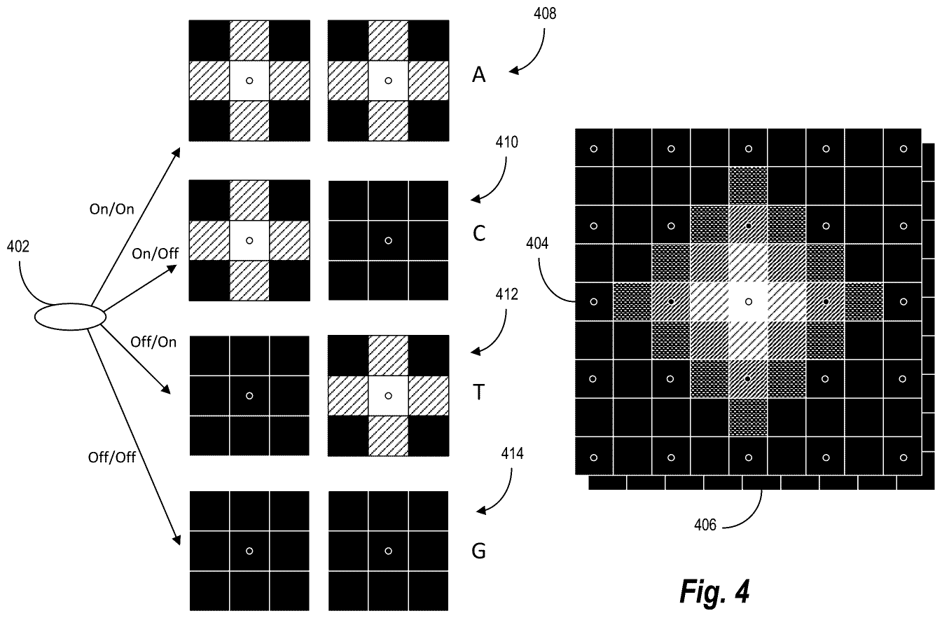

As indicated above, the crosstalk-aware-base-calling system 106 can determine nucleobase calls and corresponding illumination indicators. In accordance with one or more embodiments, FIG. 4 shows the crosstalk-aware-base-calling system 106 determining a nucleobase call and a corresponding set of illumination indicators for a cluster of oligonucleotides in different channels for a given sequencing cycle. As mentioned above, an illumination indicator indicates whether and/or to what degree a cluster provides a fluorescent response in a certain intensity channel during sequencing.

In particular, FIG. 4 shows the on/off status of sets of illumination indicators in two different intensity channels for a cluster of oligonucleotides corresponding a particular type of nucleotide base. To illustrate such an on/off status, FIG. 4 depicts light intensity in a particular frequency (e.g., frequency band) emitting or not emitting from the cluster of oligonucleotides 402 in cropped images shown in rows alongside nucleobase calls of adenine (A) 408, cytosine (C) 410, thymine (T) 412, and guanine (G) 414.

For example, as shown in FIG. 4, when making a nucleobase call of adenine (A) 408 for the cluster of oligonucleotides 402, the crosstalk-aware-base-calling system 106 determines a first set of illumination indicators indicating the cluster of oligonucleotides 402 is “on” (e.g., illuminated or emits light intensity in a particular frequency) in both a first channel captured by a first-channel image 404 and a second channel captured by a second-channel image 406. When making a nucleobase call of a cytosine (C) 410 for the cluster of oligonucleotides 402, by contrast, the crosstalk-aware-base-calling system 106 determines a second set of illumination indicators indicating the cluster of oligonucleotides 402 is “on” in the first channel captured by the first-channel image 404 and “off” (e.g., not illuminated or not emitting light intensity in a particular frequency) in the second channel captured by the second-channel image 406. When making a nucleobase call of a thymine (T) 412 for the cluster of oligonucleotides 402, the crosstalk-aware-base-calling system 106 determines a third set of illumination indicators indicating the cluster of oligonucleotides 402 is “off” in the first channel captured by the first-channel image 404 and “on” in the second channel captured by the second-channel image 406. Finally, when making a nucleobase call of a guanine (G) 414 for the cluster of oligonucleotides 402, the crosstalk-aware-base-calling system 106 determines a fourth set of illumination indicators indicating the cluster of oligonucleotides 402 is “off” in both the first channel captured by the first-channel image and the second channel captured by the second-channel image 406.

As previously discussed, the illumination status (e.g., on/active or off/inactive status) of the illumination indicator can take a couplet form or continuous form. For instance, if an illumination indicator is “on” (and emits light intensity in a particular frequency) in the intensity channel during sequencing, the “on” status can be represented by a 1. Conversely, if the illumination indicator is “off” (and does not emit light intensity in a particular frequency) in the intensity channel during sequencing, the “off” status can be represented by a 0.

Accordingly, the illumination status of a cluster of oligonucleotides in more than one channel can be represented by a set of illumination indicators. For example, the set of illumination indicators represented by [1,1] can indicate that the illumination indicator for the cluster of oligonucleotides is “on” in the first intensity channel and the second intensity channel. Additionally, the crosstalk-aware-base-calling system 106 can decode a set of illumination indicators based on the nucleobase call. For example, a set of illumination indicators for a cluster of oligonucleotides with an adenine (A) nucleotide base can be represented by [1, 1]; a cytosine (C) nucleotide base can be represented by [1,0]; a thymine (T) nucleotide base can be represented by [0, 1]; and a guanine (G) nucleotide base can be represented by [0, 0].

As previously mentioned, the illumination status of the illumination indicator can be continuous. More specifically, a given illumination indicator can indicate the degree to which a cluster of oligonucleotides is illuminated by light intensity in a particular frequency (e.g., frequency band). For example, based on the likelihood that a cluster of oligonucleotides falls within the intensity-value boundaries defined by a Gaussian mixture model, the crosstalk-aware-base-calling system 106 can determine the extent to which an illumination indicator is illuminated in a given intensity channel. Moreover, the crosstalk-aware-base-calling system 106 can determine the degree to which a continuous illumination indicator is illuminated based on the intensity values of the cluster of oligonucleotides.

Given the relationship between illumination indicators and nucleobase calls, in some embodiments, the crosstalk-aware-base-calling system 106 can update or adjust the set of illumination indicators based on a modified signal of the target cluster. For example, the crosstalk-aware-base-calling system 106 can generate a modified (and more accurate) intensity value of the target cluster by removing the inter-cluster-interference metric from the initial intensity values of the target cluster. Based on the modified intensity value of the target cluster, the crosstalk-aware-base-calling system 106 can make a different nucleobase call for the target cluster. Based on the different, and more accurate nucleobase call, the crosstalk-aware-base-calling system 106 can adjust the set of illumination indicators to represent more accurately the “on” or “off” status of the illumination indicator of the target cluster within the intensity channel. For example, in one or more embodiments, based on the initial intensity value of the target cluster, the crosstalk-aware-base-calling system 106 determines a nucleobase call of A and a set of illumination indicators for the target cluster as [1, 1]. However, based on the modified intensity values of the target cluster and corresponding nucleobase call of T, the crosstalk-aware-base-calling system 106 determines that the adjusted set of illumination indicators is [0, 1].

As discussed above, in one or more embodiments, the crosstalk-aware-base-calling system 106 can utilize the inter-cluster-interference metric to remove crosstalk from an adjacent cluster on a target cluster. In accordance with one or more embodiments, FIGS. 5A and 5B illustrate the crosstalk-aware-base-calling system 106 utilizing an equalizer system and determining an inter-cluster-interference metric representing light interference of an adjacent cluster on a target cluster and generating a modified intensity value for the target cluster based on the inter-cluster-interference metric.

As previously mentioned, the crosstalk-aware-base-calling system 106 may utilize an equalizer to estimate a modified signal. In some embodiments, the crosstalk-aware-base-calling system 106 may utilize a linear equalizer to determine an intensity value for the target cluster by processing received images. Generally, a linear equalizer is a linear filter that can be designed or optimized to filter out noise. In certain embodiments, the equalizer can convert received dispersed-over-pixels intensity energy into the received intensity value for a target cluster and an adjacent cluster by linearly weighting pixel intensities. In some embodiments, the linear filter can be applied to each cluster individually or across an entire image. In particular, FIG. 5A describes a model of the equalizer system.

When implemented on a sequencing device, in some embodiments, the crosstalk-aware-base-calling system 106 can utilize a linear equalizer to calculate the weighted sum of the intensity values of pixels that depict intensity emissions from a target cluster and one or more adjacent clusters. The equalizer may be trained to produce equalizer coefficients that are configured to mix/combine intensity values of pixels that depict intensity emissions from the target cluster and adjacent clusters in a manner that maximizes, for example, a signal-to-noise ratio.

As shown in FIG. 5A, the crosstalk-aware-base-calling system 106 can receive an input image 503 of a section of a nucleotide-sample slide. The input image can comprise of pixels depicting the intensity values of a target cluster and nearby adjacent clusters. Based on the received input image, the equalizer can gather light energy from the pixels and convert the energy to an intensity value (y_{i,c,j}) for target cluster (i) during cycle (c) in channel (j). The system model for the equalizer 505 can be modeled as y_{i,c,j}=a_{i,c,j}v_{i,c,j}+d_{i,c,j}+n_{i,c,j}. The amplification coefficient a_{i,c,j} accounts for scale variation between clusters on a nucleotide-sample slide for cycle (c), channel (j), and cluster (i). The clean intensity signal (v_{i,c,j}) accounts for an unscaled and unshifted signal for cycle (c), channel (j), and cluster (i). The DC offset (d_{i,c,j}) accounts for random noise caused by different cluster sizes, different background intensities, varying stimulation responses, varying focus, varying sensor sensitivities, and varying lens aberrations for cycle (c), channel (j), and cluster (i). The variable n(_{i,c,j}) represents additive noise for cycle (c), channel (j), and cluster (i).

Upon processing the inputs utilizing the system model for the equalizer 505, the crosstalk-aware-base-calling system 106 can determine the intensity value 507 P[x, y, c, j] for a pixel at cycle (c), location (x, y), and channel (j). As discussed below in FIG. 5B, the crosstalk-aware-base-calling system 106 can utilize the intensity of the pixels to determine a modified intensity value for the target cluster. While the described embodiment utilizes a linear equalizer to determine the intensity of pixels depicting a target cluster, other embodiments may utilize the described method with intensity detection systems and/or intensity extraction systems. In some embodiments, the crosstalk-aware-base-calling system 106 utilizes an equalizer as described by U.S. Pat. No. 11,188,778, entitled “Equalization-Based Image Processing and Spatial Crosstalk Attenuator,” by Eric Ojard et al. and U.S. application Ser. No. 18/059,326, entitled “Generating Cluster-Specific-Signal Corrections for Determining Nucleotide-Base Calls,” by Eric Ojard et al., each of which is incorporated by reference in its entirety.

As shown in FIG. 5B, and as discussed above, the crosstalk-aware-base-calling system 106 can perform the act 502 of determining a nucleobase call and illumination indicators for an adjacent cluster. As previously mentioned, the crosstalk-aware-base-calling system 106 can detect and/or measure light emitted by the adjacent cluster in a given channel during a sequencing cycle and determine intensity values for the emitted light. In some cases, based on the intensity value for the adjacent cluster, the crosstalk-aware-base-calling system 106 determines a nucleobase call for the adjacent cluster.

To illustrate such intensity-value-based base calling, in some embodiments, the crosstalk-aware-base-calling system 106 can apply an expectation maximum to a 2D Gaussian mixture model to define intensity-value boundaries corresponding to each type of nucleobase (A, C, T, or G). Based on the intensity values of light emitted by labeled nucleotides incorporated into the cluster of oligonucleotides for a given sequencing cycle, the crosstalk-aware-base-calling system 106 can determine the probability that the intensity values of the cluster of oligonucleotides fall within one of the four intensity-value boundaries corresponding to each type of nucleobase. The crosstalk-aware-base-calling system 106 can then call the nucleobase for the cluster of oligonucleotides by selecting the nucleobase with the highest probability according to the intensity-value boundaries.

As discussed above, in some embodiments, based on the nucleobase call, the crosstalk-aware-base-calling system 106 can determine a set of illumination indicators for the cluster. For instance, the crosstalk-aware-base-calling system 106 can determine the “on” and/or “off” status of an illumination indicator for an adjacent cluster in one or more intensity channels. As discussed above, in some cases, the crosstalk-aware-base-calling system 106 can represent the illumination status of the illumination indicator in couplet format. For example, if the crosstalk-aware-base-calling system 106 makes an adenine (A) nucleobase call for the adjacent cluster, the crosstalk-aware-base-calling system 106 determines that the illumination indicator for the cluster of oligonucleotides is “on” in both the first intensity channel and the second intensity channel. Based on this determination, the crosstalk-aware-base-calling system 106 can represent the on status of the cluster of oligonucleotides in both channels as the set of illumination indicators [1, 1]. As discussed with more detail below, the crosstalk-aware-base-calling system 106 can utilize the data in the set of illumination indicators to determine an inter-cluster-interference metric.

As further shown in FIG. 5B, in some embodiments, the crosstalk-aware-base-calling system 106 utilizes a signal model for a target cluster 504. More specifically, the crosstalk-aware-base-calling system 106 can utilize a function to determine the initial intensity values (P[x, y, c, j]) for a pixel (P) representing a target cluster of oligonucleotides at a location [x, y], where (x) represents the horizontal coordinate of the pixel and (y) represents the vertical coordinate of the pixel. As the signal model indicates, the initial intensity values (P[x, y, c, j]) for the pixel representing the target cluster can include the sum of the intensity values from background, the target cluster, and crosstalk emitted from adjacent clusters.

As shown in FIG. 5B, for example, the intensity values (P[x, y, c, j]) for the target cluster can be modeled as:

$$P [ x , y , c , j ] = \hat{b} [ x , y , c , j ] + \underbrace{{\sum }_{i ∈ clusters} {\hat{a}}_{i , c , j} . {\hat{v}}_{i , c , j} \times {PSF}_{J} [ x y - {y}_{ι} ]}$$

The background intensity (\hat{b}_{[x,y,c,j]}) estimates the background intensity value at a location (x, y) in the image captured during a sequencing cycle (c) in channel (j). In some cases, the estimated background intensity values can include noise or bias inherent in the genomic sample or sequencing device. For instance, in some embodiments, background intensity can increase the intensity value of a target cluster. Additionally, the function Σ_{i∈clusters} â_{i,c,j}·\hat{v}_{i,c,j}×PSF_{J}[x [symbol] y−y_{ι}] estimates the sum of intensity values from the target cluster and crosstalk from adjacent clusters.

As further shown in FIG. 5B, the sum of intensity values for the target cluster can include an estimate of the amplitude (â_{i,c,j}) of the target cluster and adjacent clusters with a cluster index (i), during a sequencing cycle (c), within an intensity channel (j). In particular, based on the intensity value of the target cluster, the crosstalk-aware-base-calling system 106 can estimate the amplitude of the target cluster and the amplitude of one or more adjacent clusters within an intensity channel. Additionally, as indicated in FIG. 5B, the crosstalk-aware-base-calling system 106 can determine the illumination indicators (\hat{v}_{i,c,j}) encoded for the target cluster and corresponding illumination indicators for one or more adjacent clusters with a cluster index (i), during a sequencing cycle (c), within an intensity channel (j).

As discussed above, in some cases, the couplet format encoded in the target cluster can be represented by a set of illumination indicators for the target cluster (e.g., [1, 1], [1, 0], [0, 1], or [0, 0]). However, as discussed above, crosstalk from the adjacent cluster with a high intensity value can inflate the intensity value of the target cluster. The increased intensity value of the target cluster could lead to an incorrect indication that the target cluster was on in the first or second intensity channels during sequencing.

FIG. 5B further illustrates that the signal model for the target cluster 504 can include an estimate of the point spread function (PSF) covering various locations (x, y) with respect to the center location (xᵢ, yᵢ) of the PSF response. For example, the crosstalk-aware-base-calling system 106 can estimate a higher PSF response for a first location closer to the center location (xᵢ, yᵢ) of the PSF response or a lower PSF response for a second location further from the center location (xᵢ, yᵢ) of the PSF response. As mentioned above, the estimated PSF can illustrate how the crosstalk spreading from an adjacent cluster interferes with the intensity values of the target cluster. More specifically, the estimated PSF can estimate the PSF response of the location of the target cluster with respect to the center of the PSF response from the adjacent cluster.

As further shown in FIG. 5B, the crosstalk-aware-base-calling system 106 can determine inter-cluster-interference metric(s) 506. As mentioned above, the inter cluster interference metric (I_{i0_i1}) can represent the light interference of one cluster (represented as i1) on another cluster (represented as i0). For instance, the inter-cluster-interference metric (I_{i0_i1}) can represent the light interference from the adjacent cluster on the target cluster.

As the function I_{i0_i1} in FIG. 5B indicates, the crosstalk-aware-base-calling system 106 can estimate the inter cluster interference metric for a given sequencing cycle (c) by multiplying the amplitude (â_{i1,c,j}) of the adjacent cluster, the illumination indicators (\hat{v}_{i1,c,j}) of the adjacent cluster, and a PSF corresponding to a location of the target cluster. In particular, the crosstalk-aware-base-calling system 106 can determine the inter-cluster-interference metric (I_{i0_i1}) in part by estimating the amplitude (â_{i1,c,j}) of the adjacent cluster (i1) at sequencing cycle (c) in channel (j). As previously mentioned, the crosstalk-aware-base-calling system 106 can estimate the amplitude (â_{i1,c,j}) of adjacent cluster (i1) based on the intensity value of the adjacent cluster.

The crosstalk-aware-base-calling system 106 can further estimate the illumination indicators (\hat{v}_{i1,c,j}) of the adjacent cluster (i1) based on the intensity value of the adjacent cluster (i1). For example, based on the high intensity value of the adjacent cluster (i1), the crosstalk-aware-base-calling system 106 determines a nucleobase call (e.g., A) for the adjacent cluster and corresponding illumination indicators (e.g., [1, 1]) for the adjacent cluster (i1) in a first intensity channel and second intensity channel.

Additionally, as shown in FIG. 5B, the crosstalk-aware-base-calling system 106 can estimate the point spread function at the location (xᵢ₀, yᵢ₀) of the target cluster (i0) with respect to PSF response of the central location (xᵢ₁, yᵢ₁) (or area) of the adjacent cluster (i1). As mentioned above, the estimated PSF corresponding to a location of the target cluster can describe how intensity values of the adjacent cluster affect the intensity values of the target cluster based on the locations of the adjacent cluster and the target cluster.

As further indicated by FIG. 5B and the corresponding functions, the crosstalk-aware-base-calling system 106 can subtract the inter-cluster metric from the sum of intensity values of the target cluster 508. In particular, the crosstalk-aware-base-calling system 106 can remove the inter-cluster-interference metric of the adjacent cluster (i1) from the sum of intensity values for the target cluster (i0). Similarly, as further depicted by FIG. 5B, the crosstalk-aware-base-calling system 106 can determine and remove the inter-cluster-interference metrics of an adjacent cluster (i2) up through adjacent cluster (in) from the sum of intensity values for the target cluster (i0). Accordingly, the crosstalk-aware-base-calling system 106 can determine and remove inter-cluster-interference metrics for multiple adjacent clusters from intensity values of a single target cluster. For instance, in some embodiments, the crosstalk-aware-base-calling system 106 can estimate and remove the inter-cluster-interference metric for the adjacent clusters with the highest intensities that are nearest to the target cluster. Moreover, the crosstalk-aware-base-calling system 106 can subtract the crosstalk originating from the adjacent cluster (i1) from any other cluster position on the flow cell.

As mentioned above, the crosstalk-aware-base-calling system 106 can iteratively determine inter-cluster-interference metrics for crosstalk of subsets of adjacent clusters on respective subsets of target clusters and remove the inter-cluster-interference metrics of the subset of adjacent clusters from the respective subset of target clusters based on the intensity-value ranges of the subset of adjacent clusters. For example, the crosstalk-aware-base-calling system 106 can determine nucleobase calls for a first subset of adjacent oligonucleotide clusters that emit the brightest signals within a top intensity-value range (e.g., top 10% brightest). In some cases, the crosstalk-aware-base-calling system 106 calls nucleobases for the brightest clusters because they have the highest likelihood of falling within the intensity-value boundary associated with one of the nucleobases (e.g., A). From the nucleobase calls of the first subset of adjacent oligonucleotide clusters, the crosstalk-aware-base-calling system 106 determines (i) illumination indicators for respective clusters from the first subset of adjacent oligonucleotide clusters and (ii) inter-cluster-interference metrics for individual adjacent clusters from the first subset of adjacent oligonucleotide clusters with respect to individual target clusters from the subset of target oligonucleotide clusters. The crosstalk-aware-base-calling system 106 further removes the inter-cluster-interference metrics of the first subset of adjacent oligonucleotide clusters from the sum of intensity values of the individual target clusters.

After such removal, the crosstalk-aware-base-calling system 106 can determine nucleobase calls, illumination indicators, and the inter-cluster-interference metrics for a second subset of adjacent clusters within a second-intensity value range (e.g., top 20-30% brightest). The crosstalk-aware-base-calling system 106 can further remove the inter-cluster-interference metrics of individual adjacent clusters of the second subset of adjacent clusters from the sum intensity value of individual target clusters from a second subset of target oligonucleotide clusters.

As further shown in FIG. 5B, after the crosstalk-aware-base-calling system 106 removes the inter-cluster-interference metric, the crosstalk-aware-base-calling system 106 can generate modified intensity values for pixel(s) depicting the target cluster 510. As indicated by FIG. 5B, for instance, the modified intensity value for a pixel depicting the target cluster (P_{[x,y,c,j]}) equals a sum of (i) a background intensity (\hat{b}_{[x,y,c,j]}) and (ii) a sum of amplitudes (â_{i,c,j}) and illumination indicators (\hat{v}_{i,c,j}) for adjacent clusters and the target cluster multiplied by a PSF. As previously indicated, the modified intensity value (\hat{P}_{[x,y,c,i]}) represents a more accurate intensity value and/or purer signal for the target cluster during a sequencing cycle. Based on the modified intensity value, the crosstalk-aware-base-calling system 106 can make a more accurate nucleobase call with minimal to no crosstalk interference from one or more adjacent clusters.

For instance, as previously mentioned, the crosstalk-aware-base-calling system 106 can calculate the probability that a signal falls within the intensity-value boundaries of a certain nucleobase (A, C, G, or T) based on Gaussian probability distributions and an expectation maximization. By removing the inter-cluster interference metric, the crosstalk-aware-base-calling system 106 can determine, based on the more accurate intensity value of the signal, a more accurate probability that the signal falls within the intensity-value boundaries of a particular nucleobase (A, C, G, or T). In some embodiments, the updated probability may change the call or prediction of the nucleobase incorporated into the cluster. In other embodiments, the updated probability may not change the call or prediction of the nucleobase incorporated into the cluster but may provide a higher base-call-quality metric (e.g., QUAL score) that the signal from the cluster falls within the intensity-value boundaries of the nucleobase that was initially called or predicted.

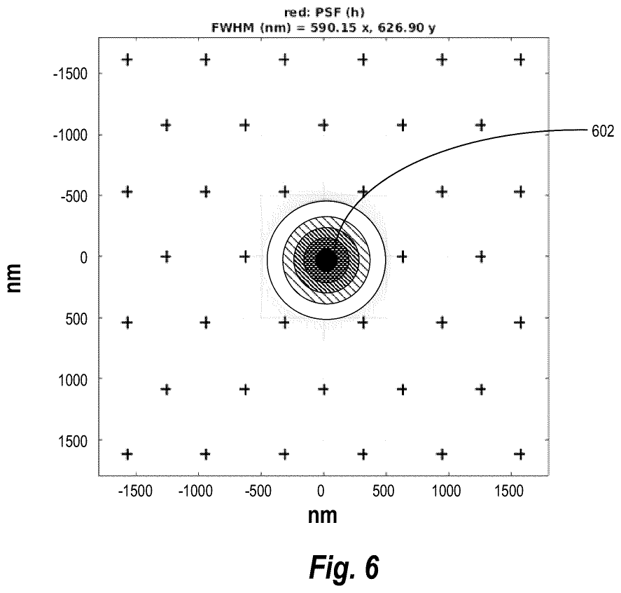

As just indicated, in some cases, the crosstalk-aware-base-calling system 106 estimates a PSF response for a section of a nucleotide-sample slide including a target cluster and adjacent clusters. In accordance with one or more embodiments, FIG. 6 illustrates the crosstalk-aware-base-calling system 106 estimating the point spread function for intensity values of a cluster.

As shown in FIG. 6, an estimated PSF can describe the response at a certain location or area with respect to the center PSF response of a point source (e.g., cluster of oligonucleotides). More specifically, FIG. 6 shows a mathematically modeled PSF response of intensity values from a cluster of oligonucleotides 602. As shown in FIG. 6, the estimated PSF of intensity values of the cluster of oligonucleotides are most concentrated (e.g., brightest) at a center location or area and decrease as the signal from the cluster of oligonucleotides moves away from the central location or area of the cluster of oligonucleotides. The crosstalk-aware-base-calling system 106 can utilize the estimated PSF response to estimate the degree of crosstalk from an adjacent cluster onto a target cluster.

In some embodiments, the PSF may be estimated by utilizing a Least-Squares (LS) or a Minimum Mean Squared Error (MMSE) method. For instance, under the Least-squares (LS) approach, the detector receives a signal (y) to determine the PSF estimate (ĥ). The received signal (y) can be expressed as y=Mh+n, where h is a complex channel impulse response (e.g., PSF response), M is a circulant training sequence matrix, and n is noise. Upon generating the training sequence matrix (M) and minimizing the squared error quantity for ĥ=argₕ min∥|y−Mh∥², the estimated least squares channel impulse response (ĥ_{LS}) may be represented by ĥ_{LS}=(M^{H}M)⁻¹M^{H}y, where, ( )^{H} and ( )⁻¹ respectively represent Hermitian and inverse matrices. Finally, given that ĥ_{LS}=(M^{H}M)⁻¹M^{H}y is the best linear unbiased estimate for the channel coefficients, the aforementioned equation may be simplified to

$$\hat{h} = \frac{1}{P} {M}^{H} y ,$$

where P represents the length of the training sequence. Thus, the PSF may be estimated based on the equation

$$\hat{h} = \frac{1}{P} {M}^{H} y .$$

In certain implementations, the crosstalk-aware-base-calling system 106 determines PSF estimates as described by Jinho Choi, Adaptive and Iterative Signal Processing in Communications (Cambridge Univ. Press 2006) or by Markku Pukkila, Channel Estimation Modeling (2000), available at http://www.comlab.hut.fi/opetus/333/presentations_2000/chan_est.pdf, both of which are incorporated herein by reference in their entirety.

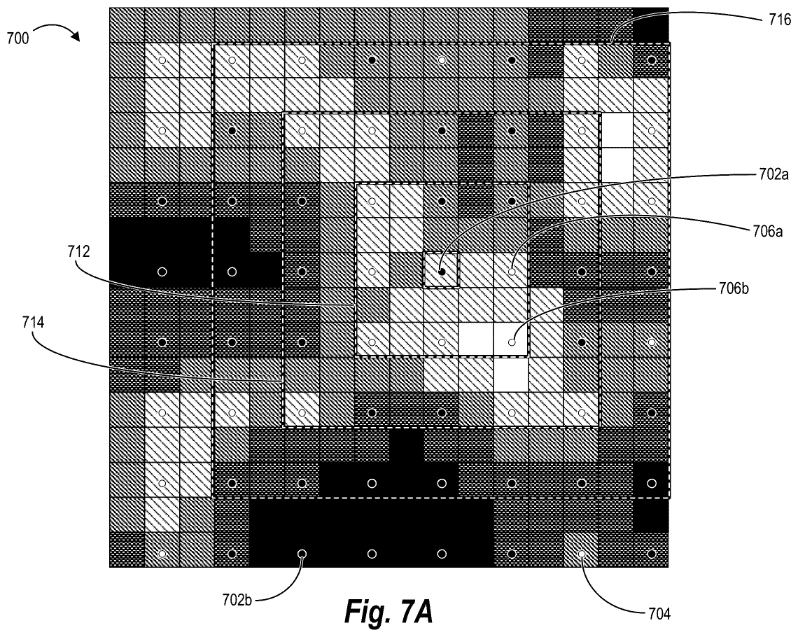

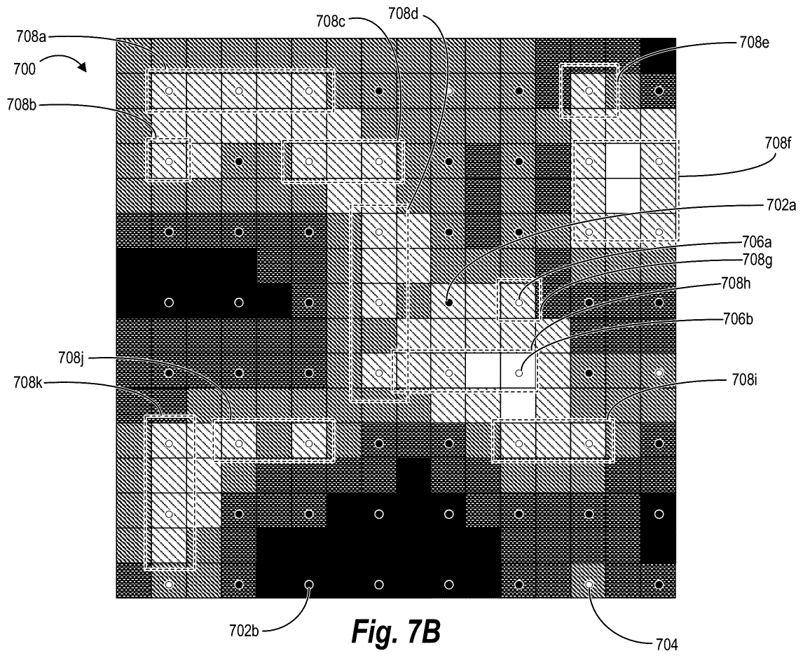

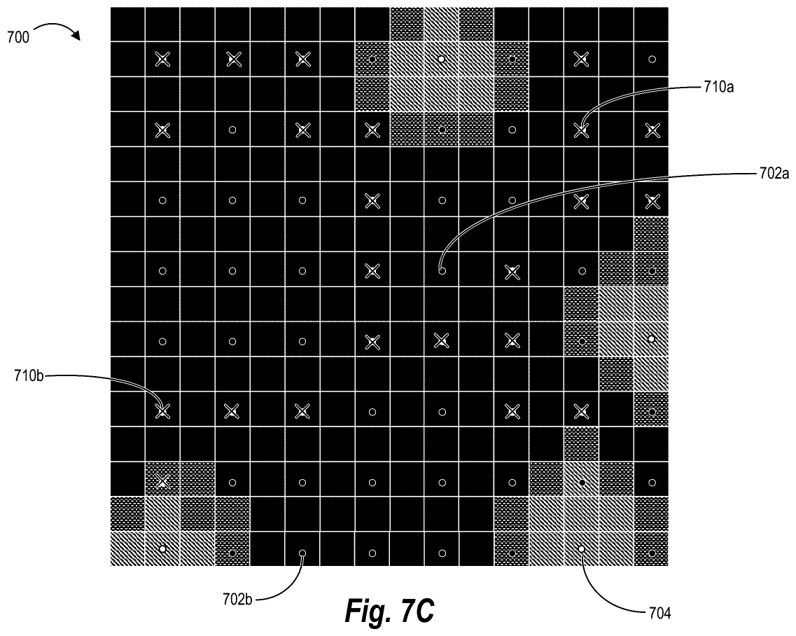

In accordance with one or more embodiments, FIGS. 7A-7C illustrate the effects of crosstalk between clusters of oligonucleotides and removing light interference for certain clusters of oligonucleotides. In particular, FIGS. 7A-7C provide simulated images of clusters of oligonucleotides on a nucleotide-sample slide and crosstalk between clusters of oligonucleotides for illustrative purposes. While the images in FIGS. 7A-7C show clusters as an evenly spaced square grid, actual clusters of oligonucleotides are not evenly dispersed on a nucleotide-sample slide. Moreover, FIGS. 7A-7C depict clusters at the center of each pixel within the square grid to more clearly illustrate the effects of crosstalk. Additionally, while the FIGS. 7A-7C illustrate a nucleotide-sample slide utilizing a square grid other embodiments of nucleotide-sample slides may utilize various shapes (e.g., diamond, hexagon, etc.).

As an overview, FIG. 7A depicts an image 700 mapping the intensity values of clusters of oligonucleotides reacting to light excitation within an intensity channel. The image 700 can represent a section of a nucleotide-sample slide (e.g., a flow cell) on which clusters of oligonucleotides have been seeded. As shown in FIG. 7A, the image 700 for the intensity channel contains several clusters of oligonucleotides and maps corresponding intensity values with pixels. In particular, the image 700 of the intensity channel uses the pixels to represent intensity values at a given location within the flow cell. The intensity values depicted by each pixel is the sum of the intensity values of the cluster of oligonucleotides, noise, and crosstalk from neighboring clusters.

FIG. 7A also depicts clusters of oligonucleotides adjacent to other clusters on a section of a nucleotide-sample slide. In particular, FIG. 7A clusters of oligonucleotides that are first adjacent, second adjacent, or third adjacent in relation to a target cluster. Adjacent clusters are first adjacent in relation to a target cluster when such adjacent clusters are positioned one cluster away from a target cluster or immediately next to the target cluster relative to other clusters. For example, the eight adjacent clusters within a first adjacent border 712 are first adjacent to the “off” cluster of oligonucleotides 702a because the eight adjacent clusters are next (and closer to) the “off” cluster of oligonucleotides 702a relative to other clusters. Relatedly, adjacent clusters are second adjacent in relation to the target cluster when such adjacent clusters are positioned two clusters away from the target cluster or after next to the target cluster. For instance, as shown in FIG. 7A, the 16 adjacent clusters within the second adjacent border 714 (and outside the first adjacent border 712) are second adjacent in relation to the “off” cluster of oligonucleotides 702a because the 16 adjacent clusters are positioned after next to the “off” cluster of oligonucleotides 702a. Similarly, adjacent clusters are third adjacent in relation to the target cluster when such adjacent clusters are positioned three clusters away from the target cluster or after, after next to the target cluster. As illustrated in FIG. 3, the 24 adjacent clusters within the third adjacent border 716 (and outside the second adjacent border 714) are third adjacent in relation to the “off” cluster of oligonucleotides 702a because the 24 adjacent clusters are positioned three clusters away from (or after, after next to) the “off” cluster of oligonucleotides 702a. As indicated above, in some embodiments, the crosstalk-aware-base-calling system 106 determines inter-cluster-interference metrics for clusters that are first adjacent, second adjacent, and/or third adjacent to a target cluster.

To represent the differing types of light emitted by various clusters, FIG. 7A depicts different patterns to represent varying intensity values for different clusters and different circle types to represent an illumination indicator for clusters in the image 700. As indicated by denser or darker and sparser or lighter patterns, darker and/or dimmer pixels represent locations and/or clusters of oligonucleotides with lower intensity values and lighter and/or brighter pixels represent locations and/or clusters of oligonucleotides with higher intensity values. Moreover, a pixel containing a black circle with a white border represents an “off” cluster of oligonucleotides that does not (or has not been detect to) emit light intensity in a particular frequency (e.g., frequency band) for a given channel, whereas a pixel containing a white circle with a black border represents an “on” cluster of oligonucleotides that emits (or has been detected to emit) light intensity in a particular frequency (e.g., frequency band) for a given channel.

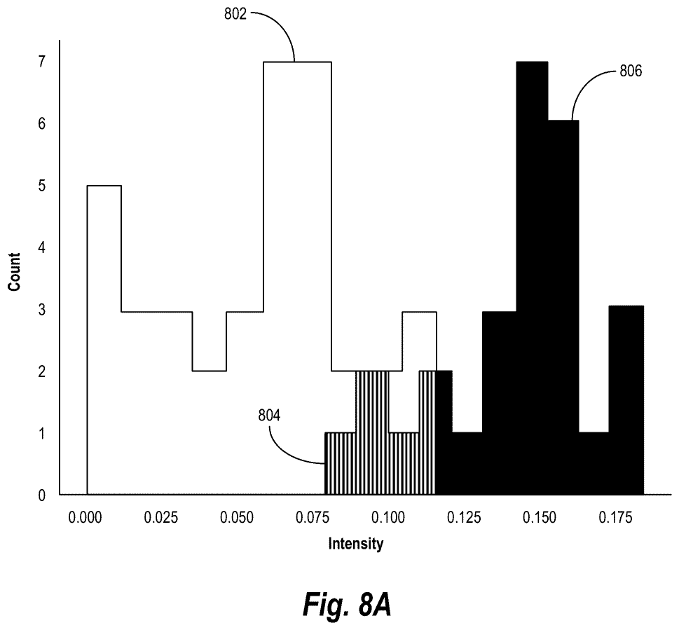

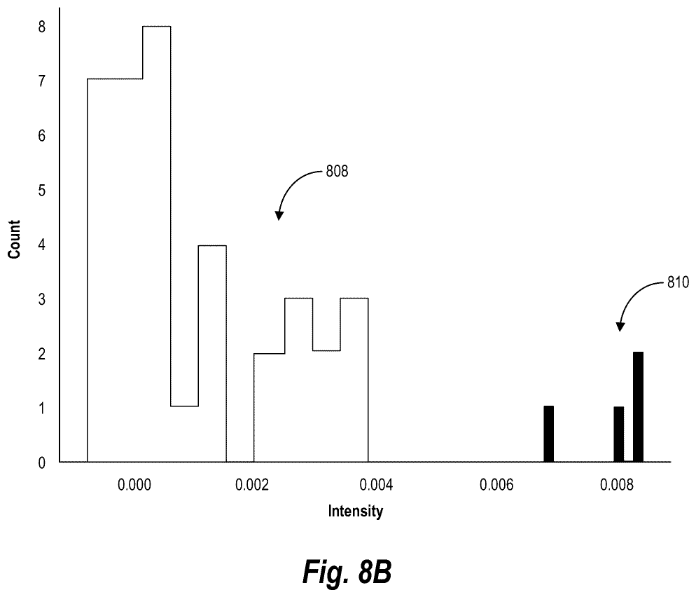

As previously discussed, it can be difficult to make a nucleobase call for “off” clusters of oligonucleotides that neighbor or are surrounded by “on” clusters of oligonucleotides with high intensity values because the crosstalk from the “on” clusters of oligonucleotides with high intensity values distorts the intensity values of the “off” clusters of oligonucleotides. In particular, FIG. 7A shows how crosstalk emitted from “on” clusters of oligonucleotides with high intensity values increases the intensity values of neighboring or adjacent “off” clusters of oligonucleotides. For example, in FIG. 7A, the “off” cluster of oligonucleotides 702a appears to be an “on” cluster of oligonucleotides because the crosstalk from bright neighboring clusters of oligonucleotides 706a and 706b makes the pixel containing the “off” cluster of oligonucleotides 702a appear brighter (e.g., increases the intensity value of the pixel). By making the pixel containing the “off” cluster of oligonucleotides 702a appear brighter, the likelihood of making an incorrect nucleobase call for the “off” cluster of oligonucleotides 702a increases. Additionally, FIG. 7A illustrates how the lower intensity values of the dim “on” clusters of oligonucleotides makes it difficult to determine the nucleobase call for the dim “on” clusters of oligonucleotides because they appear to have an intensity value that is similar to the intensity values of “off” clusters of oligonucleotides neighboring on clusters. More detail regarding, dim “on” clusters of oligonucleotides is discussed in FIG. 8A.

In accordance with one or more embodiments, FIG. 7B illustrates the crosstalk-aware-base-calling system 106 initially determining nucleobase calls and illumination indicators for a subset of clusters as part of an ordered approach to removing crosstalk. For instance, a subset of clusters of oligonucleotides highlighted by selection borders 708a, 708b, 708c, 708d, 708e, 708f, 708g, 708h, 708i, 708j, and 708k represent clusters of oligonucleotides that emit the highest intensity values within a top intensity-value range (e.g., top 10% or top 15%). As previously discussed, the high intensity values of such a subset of clusters of oligonucleotides allow the crosstalk-aware-base-calling system 106 to make a more confident determination of the nucleobase call. Based on the nucleobase calls for the subset of clusters of oligonucleotides highlighted by the selection borders 708a-708k, the crosstalk-aware-base-calling system 106 can more accurately determine that a cluster of oligonucleotides is “on” within the intensity channel.

While it is easier to make nucleobase calls for the subset of clusters of oligonucleotides highlighted by the selection borders 708a-708k, in some cases, these clusters of oligonucleotides generate the most crosstalk (e.g., light interference) and affect neighboring clusters of oligonucleotides with lower intensity values. For example, as shown in FIG. 7B, the “off” cluster of oligonucleotides 702a is surrounded by the subset of clusters of oligonucleotides highlighted by selection borders 708d, 708g, and 708h that emit the highest intensity values and levels of crosstalk. The crosstalk from the subset of clusters of oligonucleotides highlighted by the selection borders 708d, 708g, and 708h increases the intensity value of the “off” cluster of oligonucleotides 702a. By increasing the intensity value of the “off” cluster of oligonucleotides 702a, it is more likely that the “off” cluster of oligonucleotides 702a is given an inaccurate nucleobase call-without an effective way of removing crosstalk.

While FIGS. 7A-7B show the effects of crosstalk between clusters of oligonucleotides, FIG. 7C shows the effect of removing the crosstalk from certain clusters of oligonucleotides. As shown in FIG. 7C, the crosstalk-aware-base-calling system 106 removes the crosstalk from the subset of clusters of oligonucleotides highlighted by selection borders 708a-708k from the intensity values of various target clusters. To illustrate the intensity values of the light emitted by target clusters-without the light emitted by the subset of clusters of oligonucleotides highlighted by selection borders 708a-708k-FIG. 7C depicts the image 700 with patterns indicating that the light emitted by such a subset of clusters has been removed. Prior to removing crosstalk, and as discussed above, the crosstalk-aware-base-calling system 106 can (i) determine a nucleobase call and determine a set of illumination indicators for the subset of clusters of oligonucleotides highlighted by the selection borders 708a-708k, (ii) determine an inter-cluster-interference metric for each cluster of the subset of clusters of oligonucleotides highlighted by selection borders 708a-708k, and (iii) remove the inter-cluster-interference metric of the subset of clusters of oligonucleotides from other adjacent clusters of oligonucleotides with dimmer intensity values.

Upon removing the inter-cluster-interference metrics, the image 700 depicts more accurate intensity values of dimmer “on” and “off” clusters of oligonucleotides. For instance, the crosstalk-aware-base-calling system 106 cancels or removes the crosstalk emitted by the clusters of oligonucleotides 710a and 710b that interferes with the relatively lower intensity values of target clusters of oligonucleotides. As suggested by FIG. 7C, the intensity values of the “off” cluster of oligonucleotides 702a more clearly shows that the cluster of oligonucleotides 702a is “off” in a particular channel. Thus, the intensity values of the cluster of oligonucleotides 702a more closely resembles the intensity value of a cluster of oligonucleotides 702b-both of which do not emit light intensity in a particular frequency (e.g., frequency band) in a channel captured by the image 700.

As further shown in FIG. 7C, by removing the crosstalk from adjacent clusters of oligonucleotides with the highest intensity values on target cluster, the crosstalk-aware-base-calling system 106 determines modified intensity values for the cluster of oligonucleotides 704 and thereby clarify that the cluster of oligonucleotides 704 is “on” or emits light intensity in a particular frequency (e.g., frequency band) in the intensity channel during sequencing. Thus, the crosstalk-aware-base-calling system 106 can (i) make more accurate nucleobase calls based on the more accurate and modified intensity values of target clusters of oligonucleotides and (ii) more confidently determine that a given cluster of oligonucleotides is “on” or emits light intensity in a particular frequency (e.g., frequency band) in a given channel during a sequencing cycle.

As indicated above, the crosstalk-aware-base-calling system 106 improves the accuracy with which illumination indicators (and corresponding nucleobase calls) can be determined by determining and removing inter-cluster-interferences metrics. In accordance with one or more embodiments, FIGS. 8A-8B depict histograms of intensity values of clusters of oligonucleotides with and without crosstalk from adjacent clusters.

For instance, as shown in FIG. 8A, clusters of oligonucleotides with higher intensity values 806 depicted by the black values in the histogram represent clusters with intensity values for which accurate nucleobase calls can more easily be determined based on clearly “on” illumination indicators. The relatively higher (or brightest) intensity values reduce a likelihood of the crosstalk-aware-base-calling system 106 making an inaccurate nucleobase call due to crosstalk from an adjacent cluster. Thus, the crosstalk-aware-base-calling system 106 can more easily determine whether the clusters of oligonucleotides with relatively higher intensity values are “on.” Conversely, clusters of oligonucleotides with lower intensity values 802 depicted by the white values on the histogram represent clusters with intensity values for which accurate nucleobase calls can more easily be determined based on clearly “off” illumination indicators. Therefore, in the illustrated embodiment, the crosstalk-aware-base-calling system 106 can more easily determine that clusters of oligonucleotides with the lower intensity values 802 are “off.”

As further shown in FIG. 8A, however, the histogram comprises a region of overlapping intensity values 804 depicted by black-and-white striped values for which it is difficult to determine nucleobase calls and illumination indicators. For instance, when the increased intensity values of “off” clusters of oligonucleotides overlap with intensity values of “on” clusters of oligonucleotides, clusters with the overlapping intensity values 804 can prove difficult to determine accurate nucleobase calls and illumination indicators. As discussed above, in some cases, the crosstalk from bright, adjacent clusters of oligonucleotides increases the intensity values of dim “off” clusters of oligonucleotides and makes the “off” clusters of oligonucleotides appear “on” or exhibit intensity values that may or may not be “on.” Additionally, and as previously mentioned, some “on” clusters of oligonucleotides do not in fact emit light intensity in a particular frequency (e.g., frequency band) in an intensity channel with high intensity values and may appear “off” in the intensity channel.

In some cases, existing sequencing systems identify intensity-value thresholds for determining whether intensity values of a given cluster of oligonucleotides indicate the given cluster emits light intensity in a particular frequency (e.g., frequency band) in an intensity channel. As illustrated by FIG. 8A, however, an intensity-value threshold can do little to accurately determine whether clusters of oligonucleotides exhibiting the overlapping intensity values 804 should have an illumination indicator of “on” or “off” for the given intensity channel. Accordingly, the histogram depicted by FIG. 8A demonstrates that existing sequencing systems that use an intensity-value threshold-without an effective method of removing or reducing crosstalk-cannot accurately resolve illumination indicators and corresponding nucleobase calls for clusters of oligonucleotides exhibiting the overlapping intensity values 804.

In accordance with one or more embodiments, FIG. 8B illustrates that, by determining and removing inter-cluster-interference metrics representing crosstalk emitted from adjacent clusters of oligonucleotides, the crosstalk-aware-base-calling system 106 determines more accurate modified intensity values (and corresponding nucleobase calls) for target clusters. For instance, FIG. 8B shows the modified (or more accurate) intensity values for clusters of oligonucleotides. As shown in FIG. 8B, the modified intensity values 808 of “off” clusters of oligonucleotides represented as white values do not overlap with modified intensity values 810 of “on” clusters of oligonucleotides represented as black values. By clearly separating the modified intensity values 808 of the “off” clusters of oligonucleotides and the modified intensity values 810 of the “on” clusters of oligonucleotides, the crosstalk-aware-base-calling system 106 can apply an intensity-value range to clearly distinguish between “on” and “off” clusters of oligonucleotides and more accurately determine nucleobase calls for such clusters of oligonucleotides.

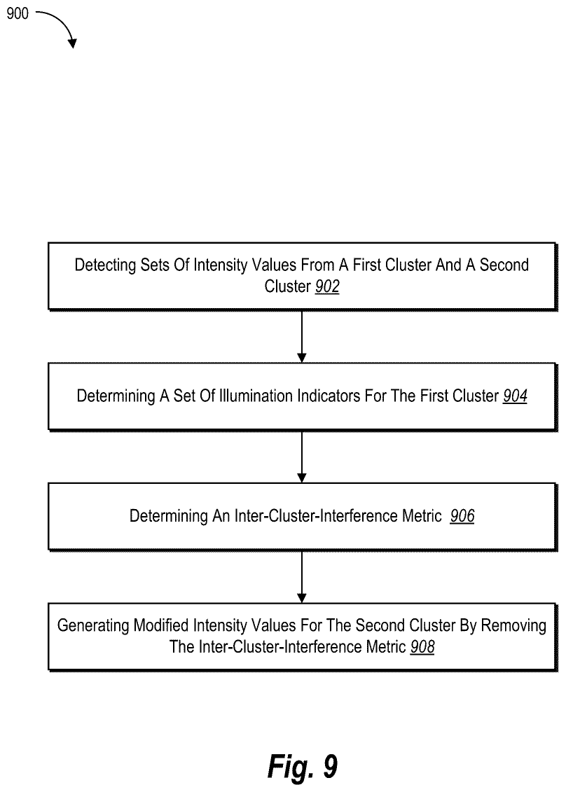

FIGS. 1-8B, the corresponding text and the examples provide a number of different methods, systems, devices, and non-transitory computer-readable media of the crosstalk-aware-base-calling system 106. In addition to the foregoing, one or more embodiments can also be described in terms of flowcharts comprising acts for accomplishing particular results, as shown in FIG. 9. FIG. 9 may be performed with more or fewer acts. Further, the acts may be performed in different orders. Additionally, the acts described herein may be repeated or performed in parallel with one another or in parallel with different instances of the same or similar acts.

FIG. 9 illustrates a flowchart of a series of acts 900 for generating a quality metric for a nucleobase call using an inter-cluster-interference metric in accordance with one or more embodiments. While FIG. 9 illustrates acts according to one embodiment, alternative embodiments may omit, add to, reorder, and/or modify any of the acts shown in FIG. 9. In some implementations, the acts of FIG. 9 are performed as part of a method. In some instances, a non-transitory computer-readable medium stores instructions thereon that, when executed by at least one processor, cause a computing device to perform the acts of FIG. 9. In some implementations, a system performs the acts of FIG. 9. For example, in one or more cases, a system includes at least one processor and a non-transitory computer readable medium comprising instructions that, when executed by the at least one processor, cause the system to perform the acts of FIG. 9.

The series of acts 900 includes an act 902 for detecting sets of intensity values from a first cluster and a second cluster. For example, the act 902 can involve detecting intensity values from a first signal from a first cluster and a second signal from a second cluster.

Additionally, the series of acts 900 includes an act 904 of determining a set of illumination indicators for the first cluster. For example, the act 904 can involve determining a nucleobase call for the first cluster and based on the nucleobase call, determining the set of illumination indicators for the first cluster.

Further, the series of acts 900 includes an act 906 of determining an inter-cluster-interference metric. For example, the act 906 can involve estimating the degree of crosstalk from a first cluster onto a second cluster by multiplying the estimated amplitude of the first cluster, set of illumination indicators of the first cluster, and the point spread function response.

The series of acts 900 further includes an act 908 of generating modified intensity values for the second cluster by removing the inter-cluster-interference metric. In particular, the act 908 can involve generating, for the sequencing cycle, a modified second set of intensity values for the second signal from the second cluster of oligonucleotides by removing the inter-cluster-interference metric from the second set of intensity values. subtracting the inter-cluster-interference metric from the sum of the intensity values of the second cluster.

In some cases, the series of acts includes additional acts of determining the set of illumination indicators based further on amplitudes for the first set of intensity values and an estimated point spread function for a section of a nucleotide-sample slide comprising the first cluster of oligonucleotides; and determining the inter-cluster-interference metric based further on the estimated point spread function.

In one or more embodiments, the series of acts 900 further includes the additional act of the estimated point spread function using a location of the second cluster of oligonucleotides or a different cluster of oligonucleotides as a point and including an area comprising the first cluster of oligonucleotides and one or more other clusters of oligonucleotides.

In some cases, the series of acts 900 includes the additional act of a first location within a nucleotide-sample slide for the first cluster of oligonucleotides is first adjacent, second adjacent, or third adjacent to a second location within the nucleotide-sample slide for the second cluster of oligonucleotides.

Further, in one or more embodiments, the series of acts 900 includes an additional act of determining, for the sequencing cycle, a nucleobase call for the first cluster of oligonucleotides based on the first set of intensity values and intensity-value boundaries for nucleobases; and determining the set of illumination indicators based further on the nucleobase call for the first cluster of oligonucleotides.

In some embodiments, the series of acts 900 also includes the additional acts of determining the nucleobase call for the first cluster of oligonucleotides based on an intensity value from the first set of intensity values corresponding to a first channel and an intensity value from the first set of intensity values corresponding to a second channel; and generating the modified second set of intensity values by subtracting values for the inter-cluster-interference metric from an intensity value from the second set of intensity values corresponding to the first channel or an intensity value from the second set of intensity values corresponding to the second channel. In some cases, the series of acts 900 include generating the modified second set of intensity values by subtracting values for the inter-cluster-interference metric from an intensity value from the second set of intensity values corresponding to the first channel or an intensity value from the second set of intensity values corresponding to the second channel. Accordingly, in certain embodiments, the inter-cluster-interference metric can be removed or cancelled from intensity values in both the first channel and the second channel.

Additionally, in other embodiments, the series of acts 900 can include the additional acts of determining a first illumination indicator indicating whether the first cluster of oligonucleotides is illuminated or not illuminated in a first channel during the sequencing cycle; and determining a second illumination indicator indicating whether the second cluster of oligonucleotides is illuminated or not illuminated in a second channel during the sequencing cycle; or determining a first continuous illumination indicator indicating a degree to which the first cluster of oligonucleotides is illuminated in the first channel during the sequencing cycle; and determining a second continuous illumination indicator indicating a degree to which the first cluster of oligonucleotides is illuminated in the second channel during the sequencing cycle.

In one or more cases, the series of acts 900 include an additional act of determining, for the sequencing cycle and based on the modified second set of intensity values, an adjusted set of illumination indicators that represents whether the second cluster of oligonucleotides is illuminated during the sequencing cycle and that differs from an initial set of illumination indicators corresponding to the second set of intensity values.

In some implementations, the series of acts 900 further includes an additional act of determining, for the sequencing cycle and based on the modified second set of intensity values, a nucleobase call for the second cluster of oligonucleotides that differs from a nucleobase corresponding to the second set of intensity values.

In additional embodiments, the series of acts 900 includes the additional acts of detecting, for the sequencing cycle, a third set of intensity values for a third signal from a third cluster of oligonucleotides; determining an additional set of illumination indicators representing whether the third cluster of oligonucleotides is illuminated during the sequencing cycle based on the third set of intensity values; determining an additional inter-cluster-interference metric estimating light interference from the third cluster of oligonucleotides on the second cluster of oligonucleotides based on the additional set of illumination indicators; and generating, for the sequencing cycle, the modified second set of intensity values for the second signal from the second cluster of oligonucleotides by removing the inter-cluster-interference metric and the additional inter-cluster-interference metric from the second set of intensity values.

Moreover, in one or more embodiments, the series of acts 900 further includes the additional acts of determining the first set of intensity values for the first signal from the first cluster of oligonucleotides is within an intensity-value range; determining the second set of intensity values for the second signal from the second cluster of oligonucleotides is not within the intensity-value range; based on the first set of intensity values being within the intensity-value range and the second set of intensity values not being within the intensity-value range, generating the modified second set of intensity values by removing, from the second set of intensity values, the inter-cluster-interference metric estimating light interference from the first cluster of oligonucleotides on the second cluster of oligonucleotides. Alternatively, the series of acts 900 includes, based on the first set of intensity values being within the intensity-value range and the second set of intensity values not being within the intensity-value range, generating the modified second set of intensity values by removing, from the second set of intensity values, the inter-cluster-interference metric estimating light interference from one or more pixels depicting the first cluster of oligonucleotides on one or more pixels depicting the second cluster of oligonucleotides.

In some cases, the series of acts 900 includes additional acts of determining, based on the first set of intensity values, a first nucleobase call for the first cluster of oligonucleotides as part of a first subset of oligonucleotide clusters having intensity values within the intensity-value range; and determining, based on the modified second set of intensity values, a second nucleobase call for the second cluster of oligonucleotides as part of a second subset of oligonucleotide clusters having intensity values not within the intensity-value range.

In one or more embodiments, the crosstalk-aware-base-calling system 106 detects the first set of intensity values by detecting, in a single channel, a first intensity value for the first signal from a first cluster of oligonucleotides; detects the second set of intensity values by detecting, in the single channel, a second intensity value for the second signal from a second cluster of oligonucleotides; and determines the set of illumination indicators by determining a single illumination indicator representing whether the first cluster of oligonucleotides is illuminated in the single channel during the sequencing cycle.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

The SBS techniques described below can utilize single-read sequencing or paired-end sequencing. In single-rea sequencing, the sequencing device reads a fragment from one end to another to generate the sequence of base pairs. In contrast, during paired-end sequencing, the sequencing device begins at one read, finishes reading a specified read length in the same direction and begins another read from the opposite end of the fragment.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the crosstalk-aware-base-calling system 106 can include software, hardware, or both. For example, the components of the crosstalk-aware-base-calling system 106 can include one or more instructions stored on a non-transitory computer readable storage medium and executable by processors of one or more computing devices (e.g., the user client device 108). When executed by the one or more processors, the computer-executable instructions of the crosstalk-aware-base-calling system 106 can cause the computing devices to perform the failure source identification methods described herein. Alternatively, the components of the crosstalk-aware-base-calling system 106 can comprise hardware, such as special purpose processing devices to perform a certain function or group of functions. Additionally, or alternatively, the components of the crosstalk-aware-base-calling system 106 can include a combination of computer-executable instructions and hardware.

Furthermore, the components of the crosstalk-aware-base-calling system 106 performing the functions described herein with respect to the crosstalk-aware-base-calling system 106 may, for example, be implemented as part of a stand-alone application, as a module of an application, as a plug-in for applications, as a library function or functions that may be called by other applications, and/or as a cloud-computing model. Thus, components of the crosstalk-aware-base-calling system 106 may be implemented as part of a stand-alone application on a personal computing device or a mobile device. Additionally, or alternatively, the components of the crosstalk-aware-base-calling system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer readable medium and executable by one or more computing devices (e.g., any of the media content access devices described herein). In general, a processor (e.g., a microprocessor) receives instructions, from a non-transitory computer readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the crosstalk-aware-base-calling system 106 and the sequencing system 104. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
detect, for a sequencing cycle, a first set of intensity values for a first signal from a first cluster of oligonucleotides and a second set of intensity values for a second signal from a second cluster of oligonucleotides;
determine a set of illumination indicators representing whether the first cluster of oligonucleotides is illuminated during the sequencing cycle based on the first set of intensity values;
determine an inter-cluster-interference metric estimating light interference from the first cluster of oligonucleotides on the second cluster of oligonucleotides based on the set of illumination indicators; and
generate, for the sequencing cycle, a modified second set of intensity values for the second signal from the second cluster of oligonucleotides by removing the inter-cluster-interference metric from the second set of intensity values.

2. The system of claim 1, further comprising instructions that, when executed by the at least one processor, cause the system to:
determine the set of illumination indicators based further on amplitudes for the first set of intensity values and an estimated point spread function for a section of a nucleotide-sample slide comprising the first cluster of oligonucleotides; and
determine the inter-cluster-interference metric based further on the estimated point spread function.

3. The system of claim 2, wherein the estimated point spread function uses a location of the second cluster of oligonucleotides or a different cluster of oligonucleotides as a point and includes an area comprising the first cluster of oligonucleotides and one or more other clusters of oligonucleotides.

4. The system of claim 1, wherein a first location within a nucleotide-sample slide for the first cluster of oligonucleotides is first adjacent, second adjacent, or third adjacent to a second location within the nucleotide-sample slide for the second cluster of oligonucleotides.

5. The system of claim 1, further comprising instructions that, when executed by the at least one processor, cause the system to:
determine, for the sequencing cycle, a nucleobase call for the first cluster of oligonucleotides based on the first set of intensity values and intensity-value boundaries for nucleobases; and
determine the set of illumination indicators based further on the nucleobase call for the first cluster of oligonucleotides.

6. The system of claim 5, further comprising instructions that, when executed by the at least one processor, cause the system to:
determine the nucleobase call for the first cluster of oligonucleotides based on an intensity value from the first set of intensity values corresponding to a first channel and an intensity value from the first set of intensity values corresponding to a second channel; and
generate the modified second set of intensity values by subtracting values for the inter-cluster-interference metric from an intensity value from the second set of intensity values corresponding to the first channel or an intensity value from the second set of intensity values corresponding to the second channel.

7. The system of claim 1, further comprising instructions that, when executed by the at least one processor, cause the system to determine the set of illumination indicators by:
determining a first illumination indicator indicating whether the first cluster of oligonucleotides is illuminated or not illuminated in a first channel during the sequencing cycle; and
determining a second illumination indicator indicating whether the second cluster of oligonucleotides is illuminated or not illuminated in a second channel during the sequencing cycle; or
determining a first continuous illumination indicator indicating a degree to which the first cluster of oligonucleotides is illuminated in the first channel during the sequencing cycle; and
determining a second continuous illumination indicator indicating a degree to which the first cluster of oligonucleotides is illuminated in the second channel during the sequencing cycle.

8. The system of claim 1, further comprising instructions that, when executed by the at least one processor, cause the system to determine, for the sequencing cycle and based on the modified second set of intensity values, an adjusted set of illumination indicators that represents whether the second cluster of oligonucleotides is illuminated during the sequencing cycle and that differs from an initial set of illumination indicators corresponding to the second set of intensity values.

9. The system of claim 1, further comprising instructions that, when executed by the at least one processor, cause the system to determine, for the sequencing cycle and based on the modified second set of intensity values, a nucleobase call for the second cluster of oligonucleotides that differs from a nucleobase corresponding to the second set of intensity values.

10. The system of claim 1, further comprising instructions that, when executed by the at least one processor, cause the system to:
detect, for the sequencing cycle, a third set of intensity values for a third signal from a third cluster of oligonucleotides;
determine an additional set of illumination indicators representing whether the third cluster of oligonucleotides is illuminated during the sequencing cycle based on the third set of intensity values;
determine an additional inter-cluster-interference metric estimating light interference from the third cluster of oligonucleotides on the second cluster of oligonucleotides based on the additional set of illumination indicators; and
generate, for the sequencing cycle, the modified second set of intensity values for the second signal from the second cluster of oligonucleotides by removing the inter-cluster-interference metric and the additional inter-cluster-interference metric from the second set of intensity values.

11. A non-transitory computer-readable medium storing instructions thereon that, when executed by at least one processor, cause a computing device to:
detect, for a sequencing cycle, a first set of intensity values for a first signal from a first cluster of oligonucleotides and a second set of intensity values for a second signal from a second cluster of oligonucleotides;
determine a set of illumination indicators representing whether the first cluster of oligonucleotides is illuminated during the sequencing cycle based on the first set of intensity values;
determine an inter-cluster-interference metric estimating light interference from the first cluster of oligonucleotides on the second cluster of oligonucleotides based on the set of illumination indicators; and
generate, for the sequencing cycle, a modified second set of intensity values for the second signal from the second cluster of oligonucleotides by removing the inter-cluster-interference metric from the second set of intensity values.

12. The non-transitory computer-readable medium of claim 11, further comprising instructions that, when executed by the at least one processor, cause the computing device to:
determine the set of illumination indicators based further on amplitudes for the first set of intensity values and an estimated point spread function for a section of a nucleotide-sample slide comprising the first cluster of oligonucleotides; and
determine the inter-cluster-interference metric based further on the estimated point spread function.

13. The non-transitory computer-readable medium of claim 12, wherein the estimated point spread function uses a location of the second cluster of oligonucleotides or a different cluster of oligonucleotides as a point and includes an area comprising the first cluster of oligonucleotides and one or more other clusters of oligonucleotides.

14. The non-transitory computer-readable medium of claim 11, further comprising instructions that, when executed by the at least one processor, cause the computing device to:
determine the first set of intensity values for the first signal from the first cluster of oligonucleotides is within an intensity-value range;
determine the second set of intensity values for the second signal from the second cluster of oligonucleotides is not within the intensity-value range; and
based on the first set of intensity values being within the intensity-value range and the second set of intensity values not being within the intensity-value range, generate the modified second set of intensity values by removing, from the second set of intensity values, the inter-cluster-interference metric estimating light interference from the first cluster of oligonucleotides on the second cluster of oligonucleotides.

15. The non-transitory computer-readable medium of claim 14, further comprising instructions that, when executed by the at least one processor, cause the computing device to:
determine, based on the first set of intensity values, a first nucleobase call for the first cluster of oligonucleotides as part of a first subset of oligonucleotide clusters having intensity values within the intensity-value range; and
determine, based on the modified second set of intensity values, a second nucleobase call for the second cluster of oligonucleotides as part of a second subset of oligonucleotide clusters having intensity values not within the intensity-value range.

16. The non-transitory computer-readable medium of claim 11, further comprising instructions that, when executed by the at least one processor, cause the computing device to:
detect the first set of intensity values by detecting, in a single channel, a first intensity value for the first signal from a first cluster of oligonucleotides;
detect the second set of intensity values by detecting, in the single channel, a second intensity value for the second signal from a second cluster of oligonucleotides; and
determine the set of illumination indicators by determining a single illumination indicator representing whether the first cluster of oligonucleotides is illuminated in the single channel during the sequencing cycle.

17. A method comprising:
detecting, for a sequencing cycle, a first set of intensity values for a first signal from a first cluster of oligonucleotides and a second set of intensity values for a second signal from a second cluster of oligonucleotides;
determining a set of illumination indicators representing whether the first cluster of oligonucleotides is illuminated during the sequencing cycle based on the first set of intensity values;
determining an inter-cluster-interference metric estimating light interference from the first cluster of oligonucleotides on the second cluster of oligonucleotides based on the set of illumination indicators; and
generating, for the sequencing cycle, a modified second set of intensity values for the second signal from the second cluster of oligonucleotides by removing the inter-cluster-interference metric from the second set of intensity values.

18. The method of claim 17, wherein a first location within a nucleotide-sample slide for the first cluster of oligonucleotides is first adjacent, second adjacent, or third adjacent to a second location within the nucleotide-sample slide for the second cluster of oligonucleotides.

19. The method of claim 17, further comprising:
determining, for the sequencing cycle, a nucleobase call for the first cluster of oligonucleotides based on the first set of intensity values and intensity-value boundaries for nucleobases; and
determining the set of illumination indicators based further on the nucleobase call for the first cluster of oligonucleotides.

20. The method of claim 19, further comprising:
determining the nucleobase call for the first cluster of oligonucleotides based on an intensity value from the first set of intensity values corresponding to a first channel and an intensity value from the first set of intensity values corresponding to a second channel; and
generating the modified second set of intensity values by subtracting values for the inter-cluster-interference metric from an intensity value from the second set of intensity values corresponding to the first channel or an intensity value from the second set of intensity values corresponding to the second channel.
